SEQUENCE LISTING (141 sequences)

SEQ ID NO: 1 - protein, 141 aa, Homo sapiens
DYKDDDDKGGGSEFEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRGSHHHHHHHHHH

SEQ ID NO: 2 - protein, 140 aa, Homo sapiens
DYKDDDDKGGGSEFEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRGSHHHHHHHHHH

SEQ ID NO: 3 - protein, 141 aa, Homo sapiens
DYKDDDDKGGGSEFEDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRGSHHHHHHHHHH

SEQ ID NO: 4 - protein, 141 aa, Homo sapiens
DYKDDDDKGGGSEFEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGGSHHHHHHHHHH

SEQ ID NO: 5 - DNA, 336 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagccgttaactgggttggtgcactcgttacgttcggacagggtaccaaggtggagatcaaacgtacg

SEQ ID NO: 6 - protein, 112 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQAVNWVGALVTFGQGTKVEIKRT

SEQ ID NO: 7 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttacttttcctggtctttttactggtttttttggtgcttatcttggtagtcttgactactggggtcaaggaaccctggtcaccgtctcctcggctagc

SEQ ID NO: 8 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFTFPGLFTGFFGAYLGSLDYWGQGTLVTVSSAS

SEQ ID NO: 9 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttacttttcctggtgcgatggatggtttttttggtgcttatcttggtagtcttgactactggggtcaaggaaccctggtcaccgtctcctcggctagc

SEQ ID NO: 10 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFTFPGAMDGFFGAYLGSLDYWGQGTLVTVSSAS

SEQ ID NO: 11 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttacttttcctggtctgtttgatgggttttttggtgcttatcttggtagtcttgactactggggtcaaggaaccctggtcaccgtctcctcggctagc

SEQ ID NO: 12 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFTFPGLFDGFFGAYLGSLDYWGQGTLVTVSSAS

SEQ ID NO: 13 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgcttttcttttccggggctatttgatgggttttttcgatcttatctggggagtcttgactactggggtcaaggaaccctggtcaccgtctcctcggctagc

SEQ ID NO: 14 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFSFPGLFDGFFRSYLGSLDYWGQGTLVTVSSAS

SEQ ID NO: 15 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttgcttttcctggtctgtttgatgggttttttcgtaattatcttggcagtcttgactactggggtcaaggaaccctggtcaccgtctcctcggctagc

SEQ ID NO: 16 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFAFPGLFDGFFRNYLGSLDYWGQGTLVTVSSAS

SEQ ID NO: 17 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgcttcgcttttcctggactttttaatgggttttttagagcttatctgggtagtcttgactactggggtcaaggaaccctggtcaccgtctcctcggctagc

SEQ ID NO: 18 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFAFPGLFNGFFRAYLGSLDYWGQGTLVTVSSAS

SEQ ID NO: 19 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttacttttcctggtctgtttgatgggttttttagggattatttaggaagtattgactactggggtcaaggaaccctggtcaccgtctcctcggctagc

SEQ ID NO: 20 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFTFPGLFDGFFRDYLGSIDYWGQGTLVTVSSAS

SEQ ID NO: 21 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttgcctttcctggtttgtttgatggtttttttcgtgattatcttggtagtattgactactggggtcaaggaaccctggtcaccgtctcctcggctagc

SEQ ID NO: 22 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFAFPGLFDGFFRDYLGSIDYWGQGTLVTVSSAS

SEQ ID NO: 23 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttgcttttcctggtctgtttgatgggttttttcgtgcgtatcttggtagtcttgactactggggtcaaggaaccctggtcaccgtctcctcggctagc

SEQ ID NO: 24 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFAFPGLFDGFFRAYLGSLDYWGQGTLVTVSSAS

SEQ ID NO: 25 - DNA, 393 bp, Homo sapiens
gaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttgcttttcctggattgttcgatggtttttttggtccttatctcggccctctagactactggggtcaaggaaccctggtcaccgtctcctcggctagc

SEQ ID NO: 26 - protein, 131 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFAFPGLFDGFFGPYLGPLDYWGQGTLVTVSSAS

SEQ ID NO: 27 - DNA, 4184 bp, Homo sapiens
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcggatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagccgttaactgggttggtgcactcgttacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 28 - DNA, 4860 bp, Homo sapiens
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttacttttcctggtctttttactggtttttttggtgcttatcttggtagtcttgactactggggtcaaggaaccctggtcaccgtctcctcggctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 29 - DNA, 4860 bp, Homo sapiens
tttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttacttttcctggtgcgatggatggtttttttggtgcttatcttggtagtcttgactactggggtcaaggaaccctggtcaccgtctcctcggctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgt

SEQ ID NO: 30 - DNA, 4860 bp, Homo sapiens
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggttcagctggtggagtctggcggtggcctggtgcagccagggggctcactccgtttgtcctgtgcagcttctggcttcaacatctcctactacttcattcactgggtgcgtcaggccccgggtaagggcctggaatgggttgcatccgtttactcctccttcggttacacctactatgccgatagcgtcaagggccgtttcactataagcgcagacacatccaaaaacacagcctacctacaaatgaacagcttaagagctgaggacactgccgtctattattgtgctcgctttacttttcctggtctgtttgatgggttttttggtgcttatcttggtagtcttgactactggggtcaaggaaccctggtcaccgtctcctcggctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 31 - protein, 18 aa, Homo sapiens
YSYPGHHSGFYSGYHGAF

SEQ ID NO: 32 - protein, 18 aa, Homo sapiens
YAYPSFYGTFFASFYGGF

SEQ ID NO: 33 - protein, 18 aa, Homo sapiens
FTFPGLFTGFFGAYLGSL

SEQ ID NO: 34 - protein, 18 aa, Homo sapiens
FAFPGHHAGFFGGHLGAF

SEQ ID NO: 35 - protein, 18 aa, Homo sapiens
YSFPGHHGGFFATYLGGF

SEQ ID NO: 36 - protein, 18 aa, Homo sapiens
FSLPGLFTGFFAGYLGAF

SEQ ID NO: 37 - protein, 18 aa, Homo sapiens
YSYPGHFTGFFSGFHGSF

SEQ ID NO: 38 - protein, 18 aa, Homo sapiens
YSFPGHHGGFFATYLGGF

SEQ ID NO: 39 - protein, 18 aa, Homo sapiens
FTFPGAMDGFFGAYLGSL

SEQ ID NO: 40 - protein, 18 aa, Homo sapiens
FTFPGDFAGFFGAYLGSL

SEQ ID NO: 41 - protein, 18 aa, Homo sapiens
FTFPGDFDGFFGAYLGSL

SEQ ID NO: 42 - protein, 18 aa, Homo sapiens
FTFPGDFRGFFGAYLGSL

SEQ ID NO: 43 - protein, 18 aa, Homo sapiens
FTFPGLFDGFFGAYLGSL

SEQ ID NO: 44 - protein, 18 aa, Homo sapiens
FTFPGPFDGFFGAYLGSL

SEQ ID NO: 45 - protein, 18 aa, Homo sapiens
FSFPGLFDGFFRSYLGSL

SEQ ID NO: 46 - protein, 18 aa, Homo sapiens
FAFPGLFDGFFRNYLGSL

SEQ ID NO: 47 - protein, 18 aa, Homo sapiens
FAFPGLFNGFFRAYLGSL

SEQ ID NO: 48 - protein, 18 aa, Homo sapiens
FTFPGLFDGFFRDYLGSI

SEQ ID NO: 49 - protein, 18 aa, Homo sapiens
FAFPGLFDGFFRDYLGSI

SEQ ID NO: 50 - protein, 18 aa, Homo sapiens
FAFPGLFDGFFRAYLGSL

SEQ ID NO: 51 - protein, 18 aa, Homo sapiens
FAFPGLFDGFFGPYLGPL

SEQ ID NO: 52 - protein, 6 aa, Homo sapiens
ISYYFI

SEQ ID NO: 53 - protein, 10 aa, Homo sapiens
SVYSSFGYTY

SEQ ID NO: 54 - protein, 9 aa, Homo sapiens
AVNWVGALV

SEQ ID NO: 55 - protein, 5 aa, Homo sapiens
SVSSA

SEQ ID NO: 56 - protein, 7 aa, Homo sapiens
SASSLYS

SEQ ID NO: 57 - protein, 54 aa, Homo sapiens
GCAGCTTCTGGCTTCAACMTCKCCTWCTWCTWCRTTCACTGGGTGCGTCAGGCC

SEQ ID NO: 58 - protein, 66 aa, Homo sapiens
GGCCTGGAATGGGTTGCAKCCRTTTWCKCCKCCTWCGSTTWCASCTWCTATGCCGATAGCGTCAAG

SEQ ID NO: 59 - protein, 90 aa, Homo sapiens
GTCTATTATTGTGCTCGCTTTACTTTTCCTGGTCTTTTTACTGGTTTTTTTGGTGCTTATCTTGGTAGTCTTGACTACTGGGGTCAAGGA

SEQ ID NO: 60 - protein, 63 aa, Homo sapiens
ACTTATTACTGTCAGCAAKCCRTTMACTKGRTTGSTSCAMTCRTTACGTTCGGACAGGGTACC

SEQ ID NO: 61 - protein, 90 aa, Homo sapiens
GTCTATTATTGTGCTCGCNNKNNKNNKNNKNNKCTTTTTACTGGTTTTTTTGGTGCTTATCTTGGTAGTCTTGACTACTGGGGTCAAGGA

SEQ ID NO: 62 - protein, 90 aa, Homo sapiens
GTCTATTATTGTGCTCGCTTTACTTTTCCTGGTNNKNNKNNKNNKTTTTTTGGTGCTTATCTTGGTAGTCTTGACTACTGGGGTCAAGGA

SEQ ID NO: 63 - protein, 90 aa, Homo sapiens
GTCTATTATTGTGCTCGCTTTACTTTTCCTGGTCTTTTTACTGGTNNKNNKNNKNNKTATCTTGGTAGTCTTGACTACTGGGGTCAAGGA

SEQ ID NO: 64 - protein, 90 aa, Homo sapiens
GTCTATTATTGTGCTCGCTTTACTTTTCCTGGTCTTTTTACTGGTTTTTTTGGTGCTNNKNNKNNKNNKNNKGACTACTGGGGTCAAGGA

SEQ ID NO: 65 - protein, 87 aa, Homo sapiens
GTCTATTATTGTGCTCGCNNKNNKNNKNNKNNKTTTACTGGTTTTTTTGGTGCTTATCTTGGTAGTCTTGACTACTGGGGTCAAGGA

SEQ ID NO: 66 - protein, 87 aa, Homo sapiens
GTCTATTATTGTGCTCGCTTTACTTTTCCTGGTCTTTTTACTGGTTTTTTTGGTNNKNNKNNKNNKNNKGACTACTGGGGTCAAGGA

SEQ ID NO: 67 - protein, 18 aa, Homo sapiens
FSFPGLFDGFFSSYLGSL

SEQ ID NO: 68 - protein, 18 aa, Homo sapiens
FTFPGLFDGFFGSYLGSF

SEQ ID NO: 69 - protein, 18 aa, Homo sapiens
FTFPGLFDGFFRAYLGSL

SEQ ID NO: 70 - protein, 18 aa, Homo sapiens
FAFPGLFEGFFRGYLGSI

SEQ ID NO: 71 - protein, 18 aa, Homo sapiens
FSFPGLFDGFFGTYLGSL

SEQ ID NO: 72 - protein, 18 aa, Homo sapiens
FSFPGLFDGFFRSYLGSL

SEQ ID NO: 73 - protein, 18 aa, Homo sapiens
FTFPGLFNGFFGEYLGSL

SEQ ID NO: 74 - protein, 18 aa, Homo sapiens
FAFPGLFDGFFRNYLGSL

SEQ ID NO: 75 - protein, 18 aa, Homo sapiens
FAFPGLFDGFFAAYLGSL

SEQ ID NO: 76 - protein, 18 aa, Homo sapiens
FSFPGMFDGFFGAYLGSL

SEQ ID NO: 77 - protein, 18 aa, Homo sapiens
FSFPGLFDGFFGAYLGSL

SEQ ID NO: 78 - protein, 18 aa, Homo sapiens
FAFPGLFDGFFGAYLGSL

SEQ ID NO: 79 - protein, 18 aa, Homo sapiens
FAFPGQFDGFFGAYLGSL

SEQ ID NO: 80 - protein, 18 aa, Homo sapiens
FSFPGLFDGFFGAYLGSI

SEQ ID NO: 81 - protein, 18 aa, Homo sapiens
FAFPGLFDGFFGAYLGSI

SEQ ID NO: 82 - protein, 18 aa, Homo sapiens
FTLPGLINGFFGAYHGSL

SEQ ID NO: 83 - protein, 18 aa, Homo sapiens
FTFPGLFNGFFGAYLGSL

SEQ ID NO: 84 - protein, 18 aa, Homo sapiens
FTFPGRFDGFFGAYLGSI

SEQ ID NO: 85 - protein, 18 aa, Homo sapiens
YTFPGLFDGFFGAYLGSL

SEQ ID NO: 86 - protein, 18 aa, Homo sapiens
FTFPGLFDGFFGAYLGSL

SEQ ID NO: 87 - protein, 18 aa, Homo sapiens
FSFPGLFDGFFRAYLGSL

SEQ ID NO: 88 - protein, 18 aa, Homo sapiens
FAFPGLFNGFFRAYLGSL

SEQ ID NO: 89 - protein, 18 aa, Homo sapiens
FAFPGLFDGFFRAYLGSL

SEQ ID NO: 90 - protein, 18 aa, Homo sapiens
FTFPGLFDGFFSAYLGSL

SEQ ID NO: 91 - protein, 18 aa, Homo sapiens
FAFPGLFDGFFAEYLGSL

SEQ ID NO: 92 - protein, 18 aa, Homo sapiens
FTFPGLFDGFFGVYLGSI

SEQ ID NO: 93 - protein, 18 aa, Homo sapiens
FTLPGLFSGFFGYYLGSL

SEQ ID NO: 94 - protein, 18 aa, Homo sapiens
FTFPGLFDGFFRDYLGSI

SEQ ID NO: 95 - protein, 18 aa, Homo sapiens
FTLPGLLDGFFRDYIGSL

SEQ ID NO: 96 - protein, 18 aa, Homo sapiens
FSFPGLFDGFFGGFLGSL

SEQ ID NO: 97 - protein, 18 aa, Homo sapiens
FSFPGLFDGFFGDYLGSL

SEQ ID NO: 98 - protein, 18 aa, Homo sapiens
FTFPGLFDGFFGDYLGSL

SEQ ID NO: 99 - protein, 18 aa, Homo sapiens
FSVPGLFDGFFRDYLGSL

SEQ ID NO: 100 - protein, 18 aa, Homo sapiens
FAFPGLFEGFFGGYLGSI

SEQ ID NO: 101 - protein, 18 aa, Homo sapiens
FTFPGLFDGFFGIYLGSL

SEQ ID NO: 102 - protein, 18 aa, Homo sapiens
FSFPGKFDGFFGSYLGSI

SEQ ID NO: 103 - protein, 18 aa, Homo sapiens
FAFPGLFDGFFSVFLGSL

SEQ ID NO: 104 - protein, 18 aa, Homo sapiens
FAFPGLFDGFFGAYLGSI

SEQ ID NO: 105 - protein, 18 aa, Homo sapiens
FAFPGLFDGFFGAYLGSL

SEQ ID NO: 106 - protein, 18 aa, Homo sapiens
FAFPGLFDGFFRDYLGSI

SEQ ID NO: 107 - protein, 18 aa, Homo sapiens
FAFPGLFDGFFRAYLGSL

SEQ ID NO: 108 - protein, 18 aa, Homo sapiens
FTFPGMFDGFFRAYLGSL

SEQ ID NO: 109 - protein, 18 aa, Homo sapiens
FTFPGLFVGFFGAYLGSL

SEQ ID NO: 110 - protein, 18 aa, Homo sapiens
FTFPGQFHGFFGDYLGSL

SEQ ID NO: 111 - protein, 18 aa, Homo sapiens
FTFPGQFDGFFGPYLGSL

SEQ ID NO: 112 - protein, 18 aa, Homo sapiens
FSFPGQFDGFFGAYLGSL

SEQ ID NO: 113 - protein, 18 aa, Homo sapiens
FTFPGQFNGFFGAYLGSL

SEQ ID NO: 114 - protein, 18 aa, Homo sapiens
FTFPGLFDGFFGSYLGSL

SEQ ID NO: 115 - protein, 18 aa, Homo sapiens
FTFPGLFGGFFRSYLGSL

SEQ ID NO: 116 - protein, 18 aa, Homo sapiens
FTFPGLFGGFFSDYLGSL

SEQ ID NO: 117 - protein, 18 aa, Homo sapiens
FTFPGLFEGFYRDYLGSL

SEQ ID NO: 118 - protein, 18 aa, Homo sapiens
FAFPGMFDGFFGDYLGSL

SEQ ID NO: 119 - protein, 18 aa, Homo sapiens
FTFPGLFDGFFRDFLGSL

SEQ ID NO: 120 - protein, 18 aa, Homo sapiens
FSSPGVFAGFFGAYIGSL

SEQ ID NO: 121 - protein, 18 aa, Homo sapiens
FTFPGLFGGFFGAYLGSL

SEQ ID NO: 122 - protein, 18 aa, Homo sapiens
STVPGLFDGFFGAYHGSL

SEQ ID NO: 123 - protein, 18 aa, Homo sapiens
YAFPGLFDGFFGAYLGSL

SEQ ID NO: 124 - protein, 18 aa, Homo sapiens
FTFPGRFDGFFGAYLGSI

SEQ ID NO: 125 - protein, 18 aa, Homo sapiens
FTFPGRFDGFFGAYLGSL

SEQ ID NO: 126 - protein, 18 aa, Homo sapiens
FSFPGLFGGFFRADLGSL

SEQ ID NO: 127 - protein, 18 aa, Homo sapiens
FTFPGLFNGFFGAYLGSL

SEQ ID NO: 128 - protein, 18 aa, Homo sapiens
FAFPGTFSGFYGAFLGSI

SEQ ID NO: 129 - protein, 18 aa, Homo sapiens
FTFPGLFSGFFGAYLGSL

SEQ ID NO: 130 - protein, 18 aa, Homo sapiens
FSFPGLFNGFFGAYIGSI

SEQ ID NO: 131 - protein, 18 aa, Homo sapiens
FTFPGLLHGFYGTYIGSL

SEQ ID NO: 132 - protein, 18 aa, Homo sapiens
YTFPGLFDGFFGKYLGSL

SEQ ID NO: 133 - protein, 18 aa, Homo sapiens
FSFPGMFDGFFGAYLGSL

SEQ ID NO: 134 - protein, 18 aa, Homo sapiens
FTFPGLFDGFFSAYLGSL

SEQ ID NO: 135 - protein, 18 aa, Homo sapiens
FTFPGLFGGFFGGYLGSL

SEQ ID NO: 136 - protein, 18 aa, Homo sapiens
YSFPGLFDGFFGAYLGSL

SEQ ID NO: 137 - protein, 18 aa, Homo sapiens
FTFPGLFAGFFGAYLGSL

SEQ ID NO: 138 - protein, 18 aa, Homo sapiens
FSFPGLFHGFFGAYLGSL

SEQ ID NO: 139 - protein, 18 aa, Homo sapiens
FAFPGLFDGFFGPYLGPL

SEQ ID NO: 140 - protein, 18 aa, Homo sapiens
FTFPGAFTGFFGAYLGSL

SEQ ID NO: 141 - DNA, 89 bp, Homo sapiens
gtctattattgtgctcgcywtrstywtsytrstywtywtrstrstywtywtrstrstywtywtrstrstywtgactactggggtcaagg

(19) United States
(12) Patent Application Publication
(10) Pub. No.: US 2014/0242095 A1
(43) Pub. Date: Aug. 28, 2014

(54) ANTIBODIES AND ANTIBODY FRAGMENTS TARGETING SIRP-ALPHA AND THEIR USE IN TREATING HEMATOLOGIC CANCERS

(71) Applicants: UNIVERSITY HEALTH NETWORK, Toronto (CA); THE HOSPITAL FOR SICK CHILDREN, Toronto (CA); THE GOVERNING COUNCIL OF THE UNIVERSITY OF TORONTO, Toronto (CA)

(72) Inventors: Jean C. Y. Wang, Toronto (CA); Jayne S. Danska, Toronto (CA); John Dick, Toronto (CA); Sachdev Sidhu, Toronto (CA); Maruti Uppalapati, Toronto (CA)

(73) Assignee: University Health Network, Toronto (CA)

(21) Appl. No.: 14/352,265

(22) PCT Filed: Oct. 19, 2012
(86) PCT No.: PCT/CA2012/000964

§ 371 Date: Apr. 16, 2014

Related U.S. Application Data
(60) Provisional application No. 61/548,817, filed on Oct. 19, 2011.

(51) Int. Cl. C07K 16/18

(52) U.S. Cl. CPC C07K 16/18
USPC 424/174.1; 530/389.7

(57) ABSTRACT

The invention relates to modulating the SIRPα—CD47 interaction in order to treat hematological cancer and compounds therefor. In particular, there is also provided SIRPα antibodies and antibody fragments, preferably used for treating hematological cancer.

### RELATED APPLICATIONS

This application claims priority from U.S. Provisional Patent Application No. 61/548,817 filed on Oct. 19, 2011.

### FIELD OF THE INVENTION

The invention relates to antibodies and antibody fragments to SIRPα, and their use in treating hematological cancer, particularly leukemia.

### BACKGROUND OF THE INVENTION

Graft failure in the transplantation of hematopoietic stem cells occurs despite donor-host genetic identity of human leukocyte antigens, suggesting that additional factors modulate engraftment. With the non-obese diabetic (NOD)-severe combined immunodeficiency (SCID) xenotransplantation model, it was found that the NOD background allows better hematopoietic engraftment than other strains with equivalent immunodeficiency-related mutations (Takenaka, K. et al. Polymorphism in Sirpa modulates engraftment of human hematopoietic stem cells. Nat. Immunol. 8, 1313-1323 (2007)). Polymorphisms in the Sirpa allele were identified and shown to be responsible for the differences in engraftment between the mouse strains analyzed. While the NOD background conferred the best support for human engraftment, mice with other polymorphisms of Sirpa could not be engrafted (i.e. NOD.NOR-Idd13.SCID). In mouse and human, Sirpa encodes for the SIRPα protein which interacts with its ligand CD47. In the hematopoietic system, SIRPα is mainly found on macrophages, dendritic cells, and granulocytes, while CD47 is present on most hematopoietic cells (Matozaki, T., Murata, Y., Okazawa, H. & Ohnishi, H. Functions and molecular mechanisms of the CD47-SIRPαlpha signalling pathway. Trends Cell Biol. 19, 72-80 (2009)). It was shown that the murine Sirpa allele is highly polymorphic in the extracellular immunoglobulin V-like domain which interacts with CD47. Thirty-seven (37) unrelated normal human controls were sequenced and 4 polymorphisms were identified, suggesting that the Sirpa allele is polymorphic in humans as it is in mice (Takenaka et al. supra).

A large body of work has shown that human acute myeloid leukemia (AML) clones are hierarchically organized and maintained by leukemia stem cells (LSC) (Wang, J. C. & Dick, J. E. Cancer stem cells: lessons from leukemia. Trends Cell Biol. 15, 494-501 (2005)). However, little is known about molecular regulators that govern LSC fate. CD47 is expressed in most human AML samples, but the level of expression on leukemic blasts varies. CD47 expression is higher on human LSCs compared to normal HSCs (Majeti, R. et al, CD47 is an adverse prognostic factor and therapeutic antibody target on human acute myeloid leukemia stem cells. Cell 138, 286 (2009) and Theocharides, A. et al, Journal of Experimental Medicine 209, 1883 (2012). Higher CD47 expression has been shown to be an independent poor prognostic factor in AML (Majeti et al., supra). Treatment of immune-deficient mice engrafted with human AML with a monoclonal antibody directed against CD47 results in reduction of leukemic engraftment in the murine bone marrow (Majeti et al., supra). However, it was not clear if this effect is specifically mediated through disruption of CD47-SIRPα interactions, as CD47 also binds to SIRPγ and to the integrin β3 subunit (Matozaki et al., supra). Recently, Danska, Dick and Wang reported that direct blockade of SIRPα binding to CD47 diminished AML engraftment, migration to distant sites and impaired engraftment in serial transplantation experiments, providing evidence that SIRPα modulates LSC function Theocharides, A. et al, Journal of Experimental Medicine 209, 1883 (2012).

WO10/30053 describes methods of treating hematological cancer comprising modulating the interaction between human Sirpa and human CD47. Applicants describe in WO10/30053 that CD47-SIRPα interaction modulates homing and engraftment of LSC in a human AML xenotransplant model.

### SUMMARY OF THE INVENTION

In an aspect, there is provided an antibody comprising at least one CDR selected from the group consisting of: CDRL1: S-V-S-S-A (SEQ ID NO. 55); CDRL2: S-A-S-S-L-Y-S (SEQ ID NO. 56); CDRL3: A-V-N-W-V-G-A-L-V (SEQ ID NO. 54); CDRH1: I-S-Y-Y-F-I (SEQ ID NO. 52); CDRH2: S-V-Y-S-S-F-G-Y-T-Y (SEQ ID NO. 53); and CDRH3: X₁-X₂-X₃-X₄-X₅-X₈-X₇-X₈-X₉-X₁₀-X₁₁-X₁₂-X₁₃-X₁₄-X₁₅-X₁₆-X₁₇-X₁₈;
wherein:
X₁ is F or Y;
X₂ is T, A or S;
X₃ is F, Y, L or V;
X₄ is P;
X₅ is G;
X₆ is L, H, F, M, Q, R, V, K, T or A;
X₇ is F, H, I, L or M;
X₈ is D, E, N, A, S, T or G;
X₉ is G;
X₁₀ is F;
X₁₁ is F or Y;
X₁₂ is G, R, A, S or T;
X₁₃ is A, S, T, G, D, E, K, Y, N or P;
X₁₄ is Y, F or H;
X₁₅ is L, H, Y or I;
X₁₆ is G;
X₁₇ is S, A, G or P; and
X₁₈ is L, F or I.

In a further aspect, there is provided the antibody described herein, for use in the treatment of hematological cancer, preferably leukemia, and further preferably acute myeloid leukemia or acute lymphoblastic leukemia.

In a further aspect, there is provided a pharmaceutical composition comprising the antibody described herein and a pharmaceutically acceptable carrier.

In a further aspect, there is provided a use of the antibody described herein, for the treatment of hematological cancer, preferably leukemia, and further preferably acute myeloid leukemia or acute lymphoblastic leukemia.

In a further aspect, there is provided a use of the antibody described herein, in the preparation of a medicament for the treatment of hematological cancer, preferably leukemia, and further preferably acute myeloid leukemia or acute lymphoblastic leukemia.

In a further aspect, there is provided a method of treating hematological cancer, preferably leukemia, and further preferably acute myeloid leukemia or acute lymphoblastic leukemia, in a subject in need of treatment, the method comprising administering a therapeutically effective amount of the antibody described herein.

In a further aspect, there is provided an isolated nucleic acid comprising a sequence that encodes the antibody described herein. In a further aspect, there is provided an expression vector comprising the nucleic acid operably linked to an expression control sequence. In a further aspect, there is provided a cultured cell comprising the vector.

### BRIEF DESCRIPTION OF FIGURES

These and other features of the preferred embodiments of the invention will become more apparent in the following detailed description in which reference is made to the appended drawings wherein:

FIG. 1 shows the complete amino sequences of the expressed SIRPα, beta and gamma proteins.

FIG. 2 shows a comparison of eluted fractions from Ni-NTA column for the purified SIRPα, beta and gamma proteins.

FIG. 3 shows binding of four clones to human SIRPαV1 and SIRPαV2 and non-specific controls.

FIG. 4 is a schematic of the plate-based binding assay for anti-SIRPα Fab.

FIG. 5 shows the binding affinity of anti-SIRPα Fab to human SIRPα-Fc fusion proteins.

FIG. 6 shows the nucleotide and amino acid sequences for (λ) SIRP29-AM3-35-VL (B) SIRP29-AM3-35-VH; (C) SIRP29-AM4-1-VH; (D) SIRP29-AM4-5-VH; (E) SIRP29-AM5-1-VH; (F) SIRP29-AM5-2-VH; (G) SIRP29-AM5-3-VH; (H) SIRP29-AM5-4-VH; (I) SIRP29-AM5-5-VH; (J) SIRP29-AM5-6-VH; and (K) SIRP29-AM5-7-VH.

FIG. 7 shows the nucleotide sequences for the (λ) SIRP29-hk-LC vector; (B) SIRP29-AM3-35-HC vector; (C) SIRP29-AM4-1-HC vector; and (D) SIRP29-AM4-5-HC vector.

FIG. 8 shows the sequences of Fabs from the 4^{th} round of affinity maturation. Only CDRH1, CDRH2, CDRH3 and CDRL3 sequences are shown. Only CDRH3 sequences vary among the clones due to the strategy used for this round of maturation

FIG. 9 shows the surface plasmon resonance measured affinities of: A) anti-SIRPα Fab and for human SIRPα-V1Fc fusion protein. B) A series of Fab made by affinity maturation of the parent clone AM4-5 for human SIRPα V1-Fc protein

FIG. 10 is a schematic of the cell-based hSIRPα binding assay.

FIG. 11 is a schematic of the quantitative assay for anti-human SIRPα-Fab binding to human SIRPα expressed on macrophages or CHO cells.

FIG. 12 shows cell-based binding assay: A) affinity comparison of anti-human SIRPα Fab 35 and hCD47-Fc for binding to human SIRPα-V1 expressed on NOR mouse macrophages, and B) calculated IC50 values for these interactions.

FIG. 13 shows the binding inhibition by three anti-SIRPa antibody format compounds (AM3-35, AM4-5 and AM4-1) of binding between CD47-Fc and hSIRPα V2 expressed on mouse macrophages.

FIG. 14 shows inhibition of hCD47-Fc binding to human SIRPα-V2 expressed on the surface of CHO cells in A) the absence or presence of two concentrations of anti-SIRPα Ab AM4-5, and, B) Escalating concentrations of five anti-SIRPα Fab made by affinity maturation of AM4-5 (see FIG. 8).

FIG. 15 shows that anti-SIRPα Ab treatment attenuates growth and spread of human primary AML cells in vivo following their transplantation into immune-deficient mice into NSG mouse recipients.

### DETAILED DESCRIPTION

In the following description, numerous specific details are set forth to provide a thorough understanding of the invention. However, it is understood that the invention may be practiced without these specific details.

Applicants describe herein antibody and antibody fragments to SIRPα obtained through successive rounds of phage display and affinity maturation.

The terms “antibody” and “immunoglobulin”, as used herein, refer broadly to any immunological binding agent or molecule that comprises a human antigen binding domain, including polyclonal and monoclonal antibodies. Depending on the type of constant domain in the heavy chains, whole antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known.

Generally, where whole antibodies rather than antigen binding regions are used in the invention, IgG and/or IgM are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains. There is essentially no preference to the use of κ or λ light chain constant regions in the antibodies of the present invention.

As will be understood by those in the art, the immunological binding reagents encompassed by the term “antibody” extend to all human antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any human antibody-like molecule that has an antigen binding region, and this term includes antibody fragments that comprise an antigen binding domain such as Fab′, Fab, F(ab′)₂, single domain antibodies (DABs), T and Abs dimer, Fv, scFv (single chain Fv), dsFv, ds-scFv, Fd, linear antibodies, minibodies, diabodies, bispecific antibody fragments and the like.

The techniques for preparing and using various antibody-based constructs and fragments are well known in the art. Diabodies, in particular, are further described in EP 404, 097 and WO 93/11161.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, T and Abs, ds-scFv, dimers, minibodies, diabodies, bispecific antibody fragments and other fragments can also be synthesized by recombinant techniques or can be chemically synthesized. Techniques for producing antibody fragments are well known and described in the art.

The human antibodies or antibody fragments can be produced naturally or can be wholly or partially synthetically produced. Thus the antibody may be from any appropriate source, for example recombinant sources and/or produced in transgenic animals or transgenic plants, or in eggs using the IgY technology. Thus, the antibody molecules can be produced in vitro or in vivo.

Preferably, the human antibody or antibody fragment comprises an antibody light chain variable region (V_{L}) that comprises three complementarity determining regions or domains and an antibody heavy chain variable region (V_{H}) that comprises three complementarity determining regions or domains. Said VL and VH generally form the antigen binding site. The “complementarity determining regions” (CDRs) are the variable loops of β-strands that are responsible for binding to the antigen. Structures of CDRs have been clustered and classified by Chothia et al. (J Mol Biol 273 (4): 927-948) and North et al., (J Mol Biol 406 (2): 228-256). In the framework of the immune network theory, CDRs are also called idiotypes.

As used herein “fragment” relating to a polypeptide or polynucleotide means a polypeptide or polynucleotide consisting of only a part of the intact polypeptide sequence and structure, or the nucleotide sequence and structure, of the reference gene. The polypeptide fragment can include a C-terminal deletion and/or N-terminal deletion of the native polypeptide, or can be derived from an internal portion of the molecule. Similarly, a polynucleotide fragment can include a 3′ and/or a 5′ deletion of the native polynucleotide, or can be derived from an internal portion of the molecule.

In an aspect, there is provided an antibody comprising at least one CDR selected from the group consisting of: CDRL1: S-V-S-S-A (SEQ ID NO. 55); CDRL2: S-A-S-S-L-Y-S (SEQ ID NO. 56); CDRL3: A-V-N-W-V-G-A-L-V (SEQ ID NO. 54); CDRH1: I-S-Y-Y-F-I (SEQ ID NO. 52); CDRH2: S-V-Y-S-S-F-G-Y-T-Y (SEQ ID NO. 53); and CDRH3: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈;
wherein:
X₁ is F or Y;
X₂ is T, A or S;
X₃ is F, Y, L or V;
X₄ is P;
X₅ is G;
X₆ is L, H, F, M, Q, R, V, K, T or A;
X₇ is F, H, I, L or M;
X₈ is D, E, N, A, S, T or G;
X₉ is G;
X₁₀ is F;
X₁₁ is F or Y;
X₁₂ is G, R, A, S or T;
X₁₃ is A, S, T, G, D, E, K, Y, N or P;
X₁₄ is Y, For H;
X₁₅ is L, H, Y or I;
X₁₆ is G;
X₁₇ is S, A, G or P; and
X₁₈ is L, F or I.

In one embodiment, X₁ is F, X₃ is F, X₁₁ is F, and X₁₈ is L.

In alternate embodiments, CDRH3 is

(SEQ ID NO. 52)
F-T-F-P-G-A-F-T-G-F-F-G-A-Y-L-G-S-L;
(SEQ ID NO. 39)
F-T-F-P-G-A-M-D-G-F-F-G-A-Y-L-G-S-L;
(SEQ ID NO. 42)
F-T-F-P-G-D-F-R-G-F-F-G-A-Y-L-G-S-L;
(SEQ ID NO. 43)
F-T-F-P-G-L-F-D-G-F-F-G-A-Y-L-G-S-L;
(SEQ ID NO. 45)
F-S-F-P-G-L-F-D-G-F-F-R-S-Y-L-G-S-L;
(SEQ ID NO. 46)
F-A-F-P-G-L-F-D-G-F-F-R-NS-Y-L-G-S-L;
(SEQ ID NO. 47)
F-A-F-P-G-L-F-N-G-F-F-R-A-Y-L-G-S-L;
(SEQ ID NO. 48)
F-T-F-P-G-L-F-D-G-F-F-R-D-Y-L-G-S-I;
(SEQ ID NO. 49)
F-A-F-P-G-L-F-D-G-F-F-R-D-Y-L-G-S-I;
(SEQ ID NO. 50)
F-A-F-P-G-L-F-D-G-F-F-R-A-Y-L-G-S-L;
or
(SEQ ID NO. 51)
F-A-F-P-G-L-F-D-G-F-F-G-P-Y-L-G-P-L.

In some embodiments, the remaining residues in any portion of the light chain variable domain, of the antibody, comprises the corresponding residues from SEQ ID NO. 14.

In some embodiments, the remaining residues in any portion of the heavy chain variable domain, of the antibody, comprises the corresponding residues from SEQ ID NO. 16.

In some embodiments, the antibody comprises at least CDRH1, CDRH2 and CDRH3.

In some embodiments, the antibody comprises all of CDRL1, CDRL2, CDRL3, CDRH1, CDRH2 and CDRH3.

In a further aspect, there is provided the antibody described herein, for use in the treatment of hematological cancer, preferably leukemia, and further preferably acute myeloid leukemia or acute lymphoblastic leukemia.

As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma.

In a further aspect, there is provided a pharmaceutical composition comprising the antibody described herein and a pharmaceutically acceptable carrier.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

In a further aspect, there is provided a use of the antibody described herein, for the treatment of hematological cancer, preferably leukemia, and further preferably acute myeloid leukemia or acute lymphoblastic leukemia.

In a further aspect, there is provided a use of the antibody described herein, in the preparation of a medicament for the treatment of hematological cancer, preferably leukemia, and further preferably acute myeloid leukemia or acute lymphoblastic leukemia.

In a further aspect, there is provided a method of treating hematological cancer, preferably leukemia, and further preferably acute myeloid leukemia or acute lymphoblastic leukemia, in a subject in need of treatment, the method comprising administering a therapeutically effective amount of the antibody described herein.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

In a further aspect, there is provided an isolated nucleic acid comprising a sequence that encodes the antibody described herein. In a further aspect, there is provided an expression vector comprising the nucleic acid operably linked to an expression control sequence. In a further aspect, there is provided a cultured cell comprising the vector.

As used herein “fusion protein” refers to a composite polypeptide, i.e., a single contiguous amino acid sequence, made up of two (or more) distinct, heterologous polypeptides which are not normally or naturally fused together in a single amino acid sequence. Thus, a fusion protein may include a single amino acid sequence that contains two entirely distinct amino acid sequences or two similar or identical polypeptide sequences, provided that these sequences are not normally found together in the same configuration in a single amino acid sequence found in nature. Fusion proteins may generally be prepared using either recombinant nucleic acid methods, i.e., as a result of transcription and translation of a recombinant gene fusion product, which fusion comprises a segment encoding a polypeptide of the invention and a segment encoding a heterologous polypeptide, or by chemical synthesis methods well known in the art. Fusion proteins may also contain a linker polypeptide in between the constituent polypeptides of the fusion protein.

As used herein, “polypeptide” and “protein” are used interchangeably and mean proteins, protein fragments, modified proteins, amino acid sequences and synthetic amino acid sequences. The polypeptide can be glycosylated or not.

The advantages of the present invention are further illustrated by the following examples. The examples and their particular details set forth herein are presented for illustration only and should not be construed as a limitation on the claims of the present invention.

### EXAMPLES

### Bacterial Expression of N-Terminal IgV Domains of SIRP Proteins

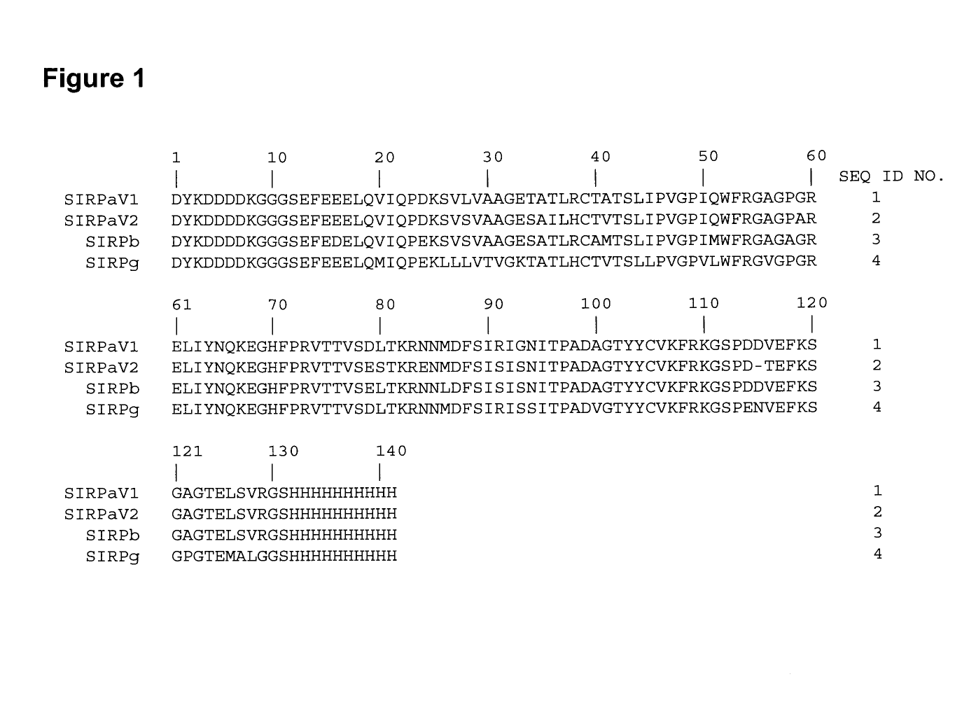

The N-terminal IgV domains of proteins SIRPαV1, SIRPαV2, SIRPβ and SIRPγ were cloned into an IPTG inducible vector pFN-OM6 with restriction sites EcoRI and BamHI, by overhang PCR using cDNA plasmids as templates (Open Biosystems Accession numbers SIRPαV1 (NM_{—}080792), SIRPαV2 (Y10375), SIRPβ (BC156609) and SIRPγ (BC064532)). The vector adds a FLAG tag at C-terminus and 10×His tag at the C-terminus of proteins. The complete amino sequences of the expressed proteins are shown in FIG. 1.

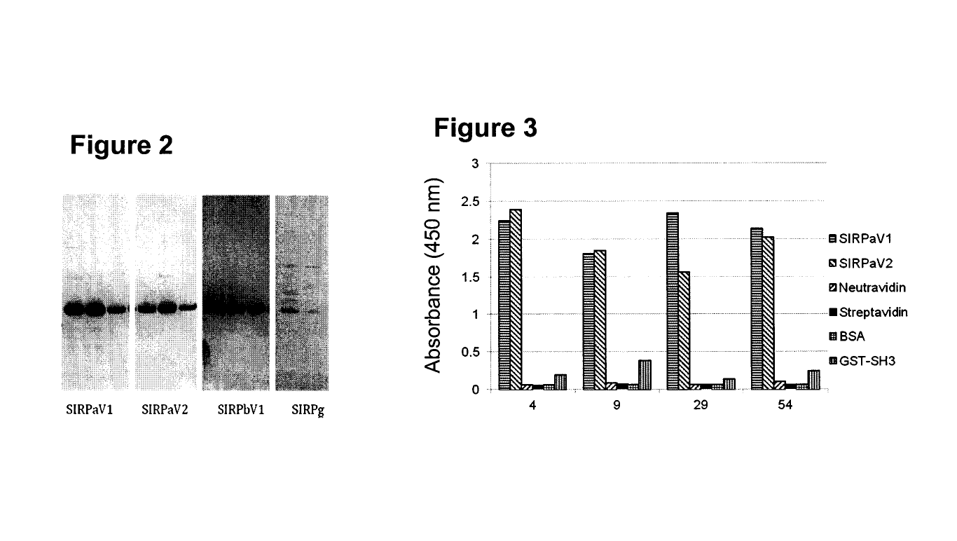

The plasmids were transformed into E. coli SS320 cells (Lucigen) and plated for single colonies. 5 ml of 2YT media with 100 ug/ml carbenicillin was inoculated and grown overnight shaking at 37° C. The overnight culture was diluted 1:250 times in 500 ml 2YT/carb media and grown until the O.D.₆₀₀ reaches 0.6. At that point, 1 mM IPTG was added to induce protein expression and the culture was incubated shaking at 37° C. for 7 hrs. The cells were harvested by centrifugation at 8000 rpm for 10 min. The protein was purified using standard Ni-NTA IMAC protocols. While the proteins SIRPαV1, SIRPαV2 and SIRPβ gave yields of nearly 3 mg/L the yield for SIRPγ was very low ˜0.15 mg/L. FIG. 2 shows the gel of purified proteins

### Phage Display Selections of Synthetic Antibody Library Against Purified SIRP Proteins

Library F is a synthetic antibody library that generated antibody binders against a variety of targets (unpublished data, Sidhu et al). Here we used Library F to select antibody binders that preferably bind to both SIRPαV1 and SIRPαV2 and not bind SIRPβ and SIRPγ. In the initial screen SIRPγ was used for negative selection.

The selection procedure is described below and is essentially the same as mentioned in previous protocols (Fellouse, F. A. & Sidhu, S. S. (2007). Making antibodies in bacteria. Making and using antibodies (Howard, G. C. & Kaser, M. R., Eds.), CRC Press, Boca Raton, Fla. and Tonikian, R., Zhang, Y., Boone, C. & Sidhu, S. S. (2007)). Identifying specificity profiles for peptide recognition modules from phage-displayed peptide libraries. Nat Protoc 2, 1368-86) with some minor changes. The media and buffer recipes are the same as in previous protocols.
1. Coat NUNC Maxisorb plate wells with 100 μl of SIRPαV1 or SIRPαV2 (5 μg/ml in PBS) for 2 h at room temperature. Coat 10 wells for selection.
2. On a separate plate coat 12 wells with SIRPγ (10 ug/ml in PBS) for 2 hrs at room temperature. This plate is for preclearing the binders to SIRPγ and the FLAG and His-tags.
3. Remove the coating solution and block for 1 h with 200 μl of PBS, 0.2% BSA. Also block the SIRPγ coated wells.
4. Remove the block solution from the pre-incubation (SIRPγ) plate and wash four times with PT buffer.
5. Add 100 μl of library phage solution (precipitated and resuspended in PBT buffer to a concentration of 10¹³ cfu/ml) to each blocked wells. Incubate at room temperature for 1 h with gentle shaking.
6. Remove the block solution from selection plate and wash four times with PT buffer.
7. Transfer library phage solution from pre-incubation plate to selection plate and let bind for 2 hrs at room temperature
8. Remove the phage solution and wash 10 times with PT buffer
9. To elute bound phage from selection wells, add 100 μl of 100 mM HCl. Incubate 5 min at room temperature. Transfer the HCl solution to a 1.5-ml microfuge tube. Adjust to neutral pH with 11 μl of 1.0 M Tris-HCl, pH 11.0.
10. Add 250 μl eluted phage solution to 2.5 ml of actively growing E. coli XL1-Blue (OD₆₀₀<0.8) in 2YT/tet medium. Incubate for 20 min at 37° C. with shaking at 200 rpm.
11. Take a 10 μl aliquot of infected cells and titer the cells by plating 10 fold serial dilutions.
12. Add M13KO7 helper phage to a final concentration of 10¹⁰ phage/ml. Incubate for 45 min at 37° C. with shaking at 200 rpm.
13. Transfer the culture from the antigen-coated wells to 25 volumes of 2YT/carb/kan medium and incubate overnight at 37° C. with shaking at 200 rpm.
14. Isolate phage by precipitation with PEG/NaCl solution, resuspend in 1.0 ml of PBT buffer
15. Repeat the selection cycle for 4 rounds by alternating the coated antigen between SIRPαV1 and SIRPαV2.

### Screening of Single-Clones by Direct Binding ELISA

96 clones were screened from 4^{th} round selection phage pool using protocols described previously (Fellouse et al. and Tonikian et al.). Four clones were identified that bind SIRPαV1 and SIRPαV2 specifically (see FIG. 3). In later tests it was found that only clone#29 bound to the glycosylated SIRPαV1 and SIRPαV2 expressed in HEK293 cells. Therefore only clone 29 was carried forward.

### First Round Affinity Maturation

CDRH3 usually has the major contribution towards binding affinity and was therefore chosen as the starting point for affinity maturation. Each residue in CDRH3 was randomized such that the original residue and three similar amino acids can occur at each position. The table below shows the substitutions

| | Homolog codon | |
|---|---|---|
| Amino acid | (IUB codes) | Mutants |
| Tyrosine (Y) | YWT | Leu, His, Phe, Tyr |
| Serine (S) | RST | Thr, Ser, Ala, Gly |
| Glycine (G) | RST | Thr, Ser, Ala, Gly |
| Alanine (A) | RST | Thr, Ser, Ala, Gly |
| Phenylalanine (F) | YWT | Leu, His, Tyr, Phe |
| Tryptophan (W) | TKS | Phe, Leu, Cys, Trp |
| Histidine (H) | YWT | Phe, Leu, Tyr, His |
| Praline (P) | SYT | Leu, Val, Ala, Pro |
| Valine (V) | NTT | Leu, Phe, Ile, Val |
| Leucine (L) | NTT | Leu, Phe, Ile, Val |
| Isoleucine (I) | NTT | Leu, Phe, Ile, Val |

A stop codon was introduced in CDRH3 of clone 29 to make a template for mutagenesis. The stop template is necessary since the mutagenesis is not 100% efficient and creates a large bias for the parent clone in the library.

Single-stranded DNA template was prepared from the stop template. The following mutagenic oligonucleotide was then used to construct a library of mutants by site-directed mutagenesis (Kunkel, T. A., Roberts, J. D. & Zakour, R. A. (1987). Rapid and efficient site-specific mutagenesis without phenotypic selection. Methods Enzymol 154, 367-82).

(SEQ ID NO. 141)
5′-GTC TAT TAT TGT GCT CGC YWT RST YWT SYT RST
YWT YWT RST RST YWT YWT RST RST YWT YWT RST RST
YWT GAC TAC TGG GGT CAA GG-3′

A library of 2×10⁹ variants was generated and selections were done as described above with these following conditions

| | Round 1 | Round 2 | Round 3 |
|---|---|---|---|
| Coated | SIRPαV1-5 μg/ml | SIRPαV2-5 μg/ml | SIRPαV1-1 μg/ml + |
| Antigen | | | SIRPαV2-1 μg/ml |
| Washes | 8 washes | 12 washes | 16 washes |
| Nega- | 5 μg/ml | 5 μg/ml | 5 μg/ml |
| tive | neutravidin | neutravidin | neutravidin |
| selec- | coated plate | coated plate | coated plate |
| tion | | | |

Competitive ELISA was used to screen 48 clones from the 3^{rd} round selection pool. The strongest binding clone 29-AM2-2 was chosen as the lead for further optimization. This round of affinity maturation resulted in roughly 10-15 times increase in affinity.

| Selected Sequences from Round1 | | SEQ |
|---|---|---|
| affinity maturation | | ID. |
| Clone#29 | Y S Y P G H H S G F Y S G Y H G A F | 31 |
| WT | | |
| 29-AM2-1 | Y A Y P S F Y G T F F A S F Y G G F | 32 |
| 29-AM2-2,6 | F T F P G L F T G F F G A Y L G S L | 33 |
| 29-AM2- | F A F P G H H A G F F G G H L G A F | 34 |
| 4,7,8 | | |
| 29-AM2-5 | Y S F P G H H G G F F A T Y L G G F | 35 |
| 29-AM2-9 | F S L P G L F T G F F A G Y L G A F | 36 |
| 29-AM2-10 | Y S Y P G H F T G F F S G F H G S F | 37 |
| 29-AM2-12 | Y S F P G H H G G F F A T Y L G G F | 38 |

### Second Round Affinity Maturation

For the second round, CDRs H1, H2 and L3 were randomized with a similar approach. However due to the large number of residues involved, each residue was randomized only with one homolog. This enables better sampling of the sequence space in a library of ˜10¹⁰ mutants.

The anti-MBP scaffold (Library F scaffold) template was used to construct the library using the following site directed mutagenesis oligos for converting the template into Clone#29 variants. The approach does not require the construction of stop template.

H1 Oligo
(SEQ ID NO. 57)
gcagcttctggcttcaac MTC KCC TWC TWC TWC RTT cactggg
tgcgtcaggcc
H2 Oligo
(SEQ ID NO. 58)
ggcctggaatgggttgca KCC RTT TWC KCC KCC TWC GST TWC
ASC TWC tatgccgatagcgtcaag
H3 Oligo (same residues as parent 29-AM2-2)
(SEQ ID NO. 59)
gtctattattgtgctcgc ttt act ttt cct ggt ctt ttt act
ggt ttt ttt ggt gct tat ctt ggt agt ctt
gactactggggtcaagga
L3 Oligo
(SEQ ID NO. 60)
acttattactgtcagcaa KCC RTT MAC TKG RTT GST SCA MTC
RTT acgttcggacagggtacc

A library of 1×10⁹ transformants was constructed and selections were done under the following conditions. At this point glycosylated SIRPα proteins were used for selection

| | | antig. | | |
|---|---|---|---|---|
| | | conc. | | |
| | Antigen | (μg/ml) | Washes | Pre-absorption |
| Round 1 | hSIRPαV1-Fc | 5 | 8 | CD47 (5 μg/ml), 1-2 h |
| | (2.76 mg/ml) | | | |
| Round 2 | hSIRPαV2-Fc | 5 | 10 | CD47 (5 μg/ml), 1-2 h |
| | (4.00 mg/ml) | | | |
| Round 3 | hSIRPαV1-Fc | 2 | 12 | CD47 (5 μg/ml), 1-2 h |
| | (2.76 mg/ml) | | | |
| Round 4 | hSIRPαV2-Fc | 1 | 12 | CD47 (5 μg/ml), 1-2 h |
| | (4.00 mg/ml) | | | |

48 clones were screened and ranked by competitive ELISA. The top three Fabs were expressed in bacteria using phoA promoter in CRAP media (after introduction of a stop codon upstream of p3 protein to convert the phagemid to an expression vector).

### Anti-hSIRPα Fab Displays High Affinity for Human Target Protein

Two anti-SIRPα Fab SIRP29-AM3-35 and SIRP29-AM3-63 (Fab 35, and Fab 63) obtained from our synthetic antibody library screen were tested for binding to two different human SIRPα-IgV domains (V1, V2). These variants represent the most common alleles in human populations (Danska et al, unpublished).

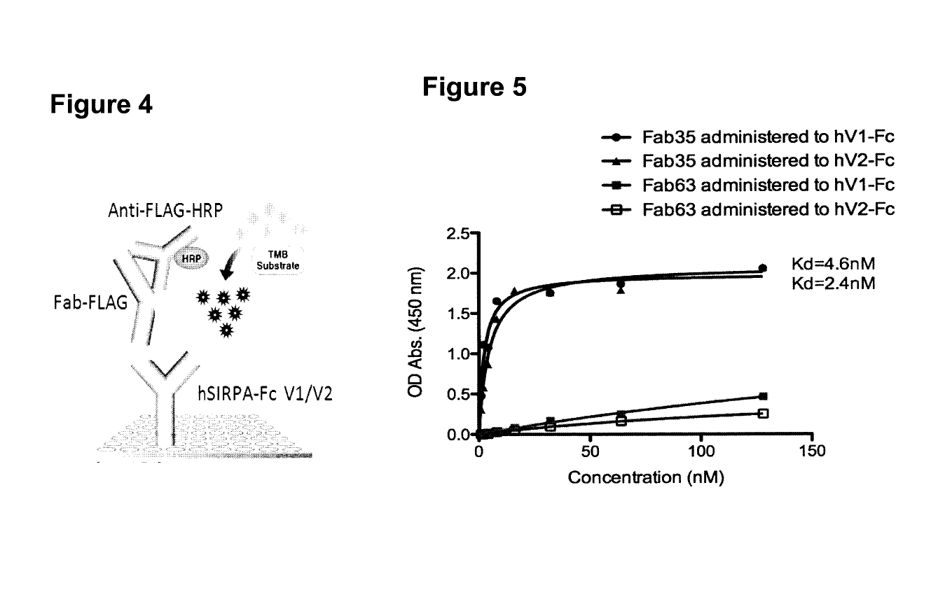

96-well microtiter plate wells were coated with human SIRPα (IgV)-Fc (V1 or V2) fusion proteins (2-5 μg/ml each) for 2 h at room temperature. After blocking with 1% (w/v) bovine serum albumin for 1 hr at room temperature, the wells were incubated with FLAG labeled anti-human SIRPα Fabs for 45 min. After washing, the coated wells were incubated with HRP-conjugated mouse monoclonal anti-FLAG antibody. Fabs binding to human SIRPα protein were detected by assaying HRP activity using the substrate 3,3′,5,5′ tetramethylbenzidine (TMB) (FIG. 4).

Fab 63 showed relatively poor binding to the target. In contrast, Fab 35 displayed low nM affinities for both forms of the human SIRPα IgV domain (FIG. 5). Fab 35 (full designation SIRP29-AM3-35) (F-T-F-P-G-A-F-T-G-F-F-G-A-Y-L-G-S-L (SEQ ID NO. 140)) was then selected as a lead antibody for further work.

### Third Round Affinity Maturation

The strategy for this round of affinity maturation is to scan the loop in stretches of 4 amino acid with NNK codon (all 20 amino acids allowed), while the other residues were kept constant. This would allow us to sample the sequence space completely for all positions and thereby replace key residues causing lower expression. In another approach, the loop was truncated by one amino acid at either end while randomizing a stretch of 5 amino acids. See below for sequences of mutagenic oligos.

Library 1 (loop length same)
(SEQ ID NO. 61)
gtctattattgtgctcgc nnk nnk nnk nnk nnk ctt ttt act ggt ttt ttt ggt gct tat ctt ggt agt
ctt gactactggggtcaagga
(SEQ ID NO. 62)
gtctattattgtgctcgc ttt act ttt cct ggt nnk nnk nnk nnk ttt ttt ggt gct tat ctt ggt agt ctt
gactactggggtcaagga
(SEQ ID NO. 63)
gtctattattgtgctcgc ttt act ttt cct ggt ctt ttt act ggt nnk nnk nnk nnk tat ctt ggt agt ctt
gactactggggtcaagga
(SEQ ID NO. 64)
gtctattattgtgctcgc ttt act ttt cct ggt ctt ttt act ggt ttt ttt ggt gct nnk nnk nnk nnk nnk
gactactggggtcaagga
Library 1 (truncated loop)
(SEQ ID NO. 65)
gtctattattgtgctcgc nnk nnk nnk nnk nnk ttt act ggt ttt ttt ggt gct tat ctt ggt agt ctt
gactactggggtcaagga
(SEQ ID NO. 66)
gtctattattgtgctcgc ttt act ttt cct ggt ctt ttt act ggt ttt ttt ggt nnk nnk nnk nnk nnk
gactactggggtcaagga

The library was constructed using the anti-MBP template and keeping the rest of the CDRs same as in the parent clone 29-AM3-35. The molecular diversity of Library 1 was 2×10¹⁰ and Library 2 was 4×10¹⁰.

It was observed that clone 29-AM3-35 also bound to NOD mouse SIRPα, although with 10 times lower affinity. Since the antibody will be tested in mouse models, it might be useful to generate clones with higher affinity to NOD-SIRPα. Therefore selections were done in a similar manner as previously alternating between human SIRPαV1 or SIRPαV2 and in parallel against NOD-SIRPα.

The selections with alternating antigens did not work due the high percentage of misfolded proteins in library. A few hits were generated against NOD-SIRPα. The selections conditions for NOD-SIRPα are shown below

| | Antigen | Conc | washes | -ve selection |
|---|---|---|---|---|
| Round1 | NOD-SIRPα-Fc | 5 μg/ml | 8 | Preabsorption on |
| | | | | 10 μg/ml GST |
| Round2 | NOD-SIRPα-Fc | 5 μg/ml | 8 | Preadsorption on either |
| | | | | 10 μg/ml Neutravidin |
| Round3 | NOD-SIRPα-Fc | 5 μg/ml | 10 | Preadsorption on |
| | | | | 10 μg/ml Streptavidin |

Competitive ELISA revealed that 3 clones (29-AM4-1,4 and 5) had a two-fold improvement in affinity to NOD-SIRPα while having a similar affinity to human SIRPαV1 and V2 when compared to parent 29-AM3-35.

| | | Seq |
|---|---|---|
| Selected Sequences from | | ID |
| Round3 affinity maturation | | NO. |
| 29-AM4-1 | F T F P G A M D G F F G A Y L G S L | 39 |
| 29-AM4-2 | F T F P G D F A G F F G A Y L G S L | 40 |
| 29-AM4-3 | F T F P G D F D G F F G A Y L G S L | 41 |
| 29-AM4-4 | F T F P G D F R G F F G A Y L G S L | 42 |
| 29-AM4-5 | F T F P G L F D G F F G A Y L G S L | 43 |
| 29-AM4-6 | F T F P G P F D G F F G A Y L G S L | 44 |

It appears that several residues in CDRH3 form secondary structure and lead to misfolding when mutated.

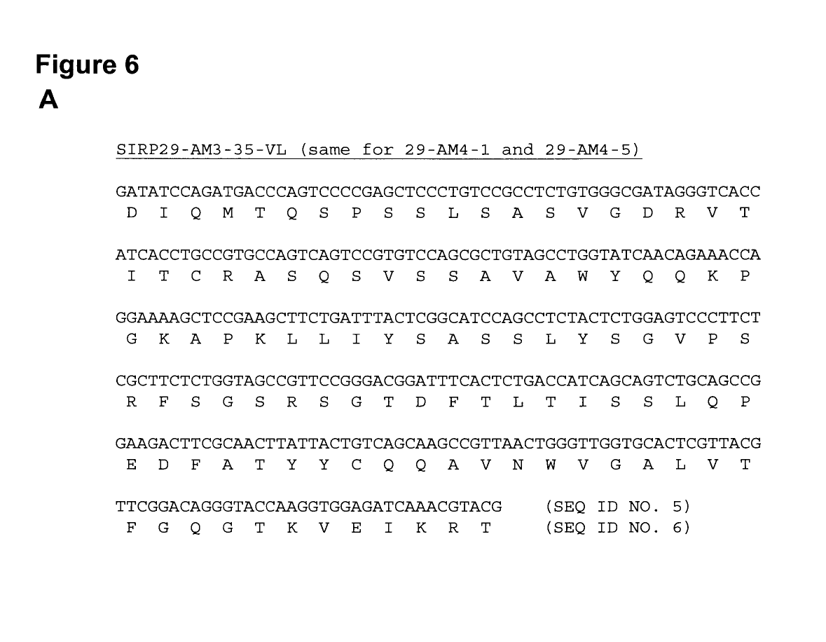

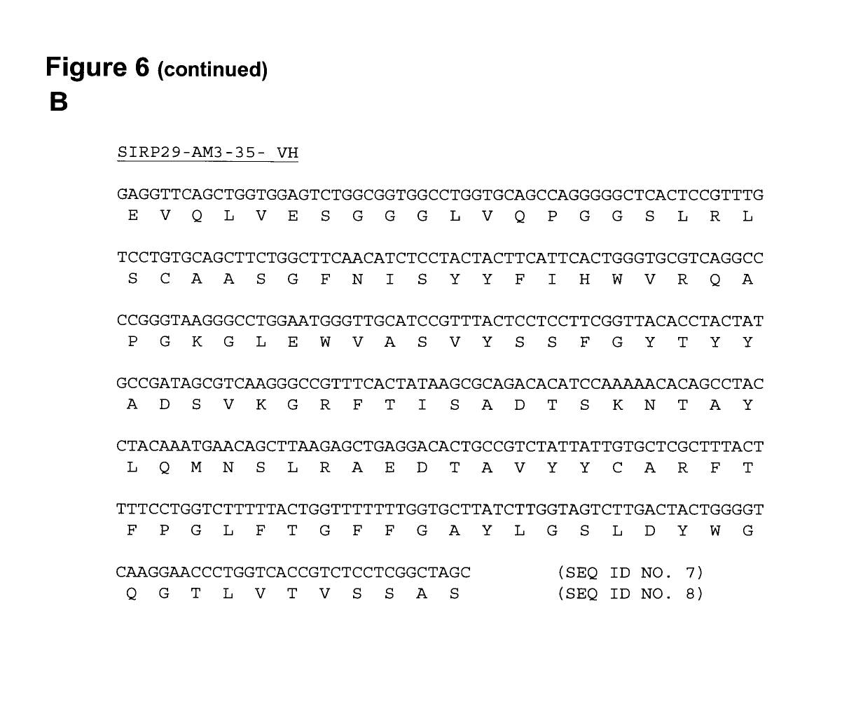

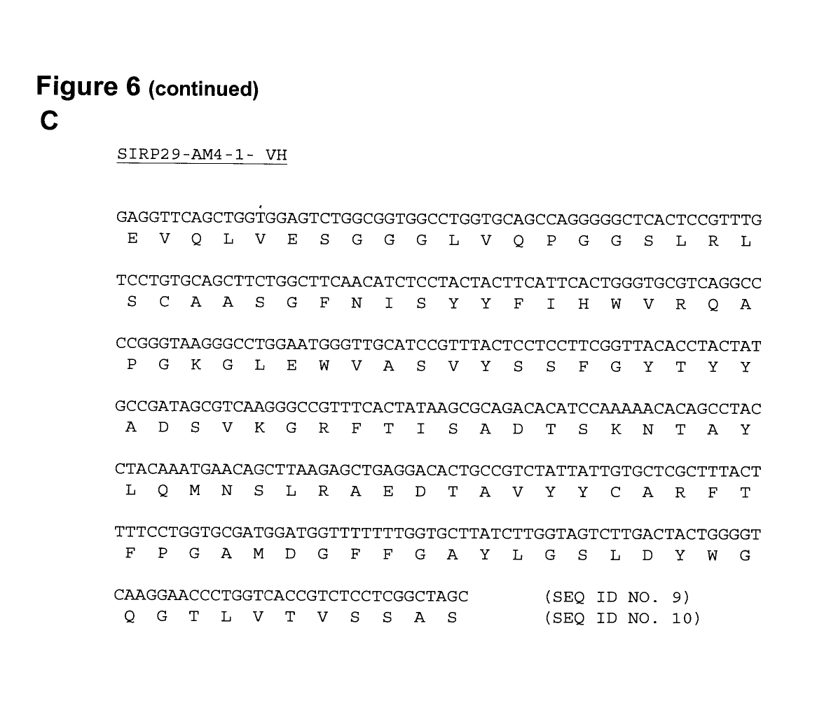

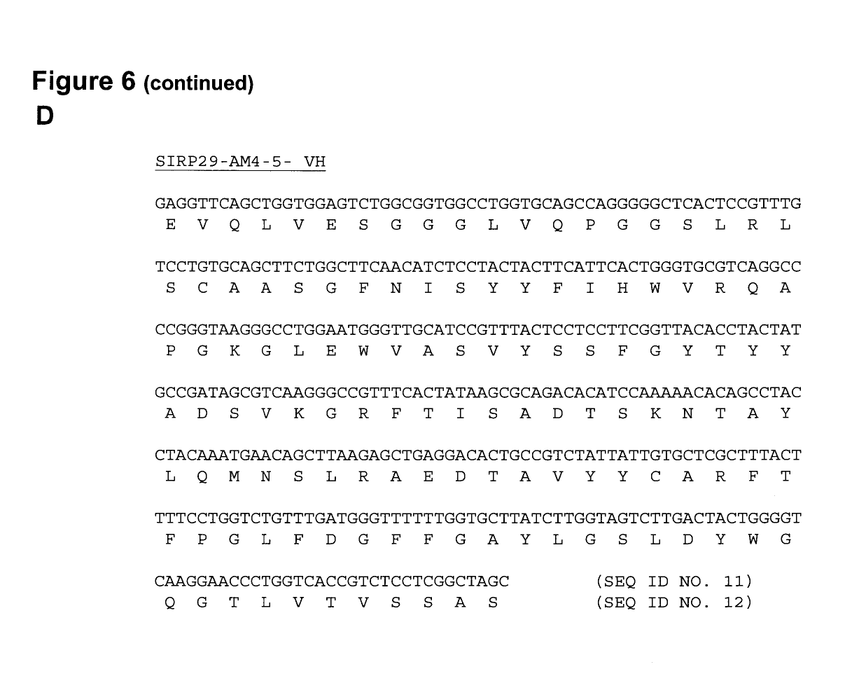

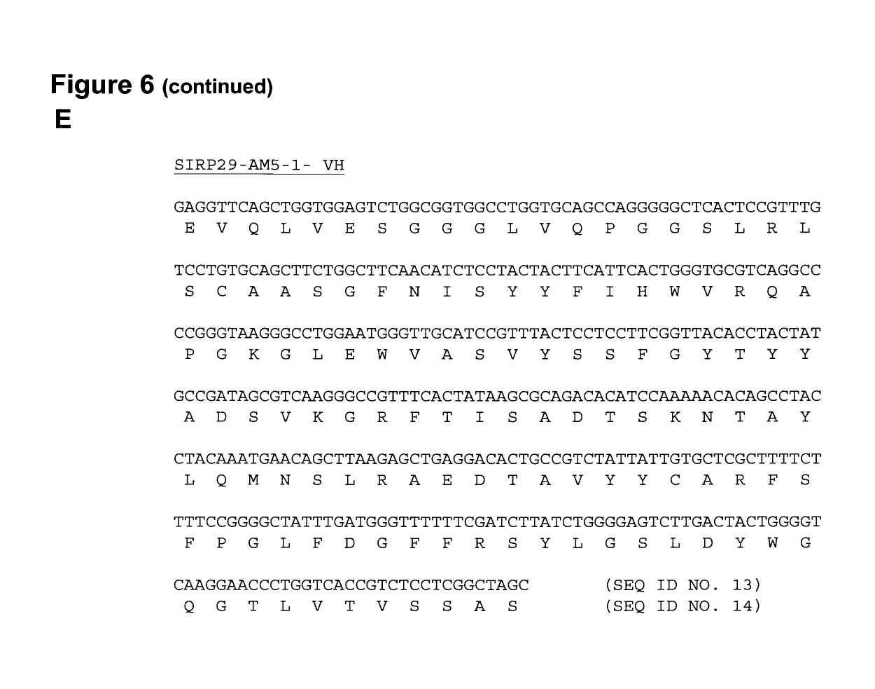

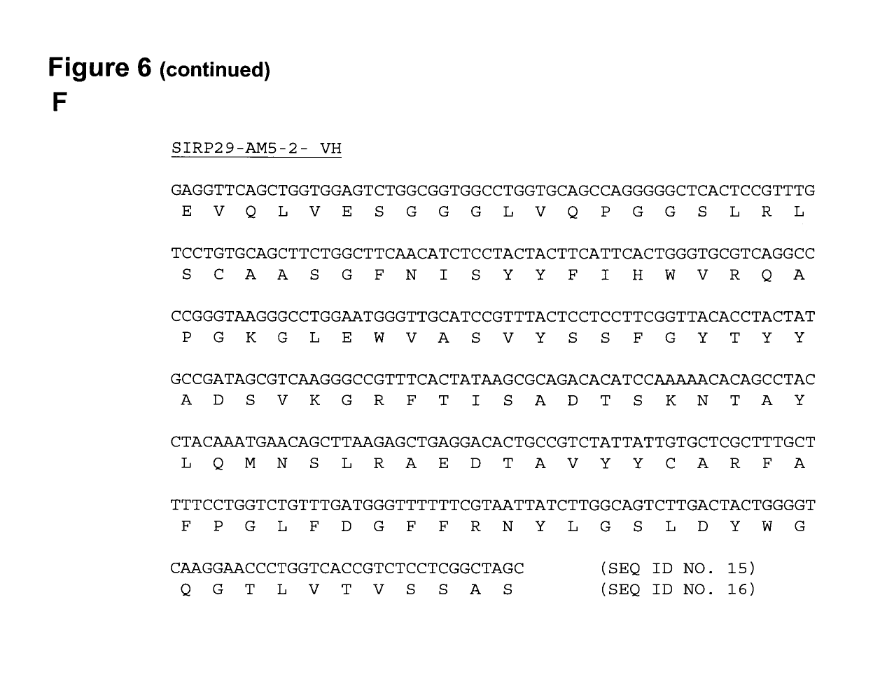

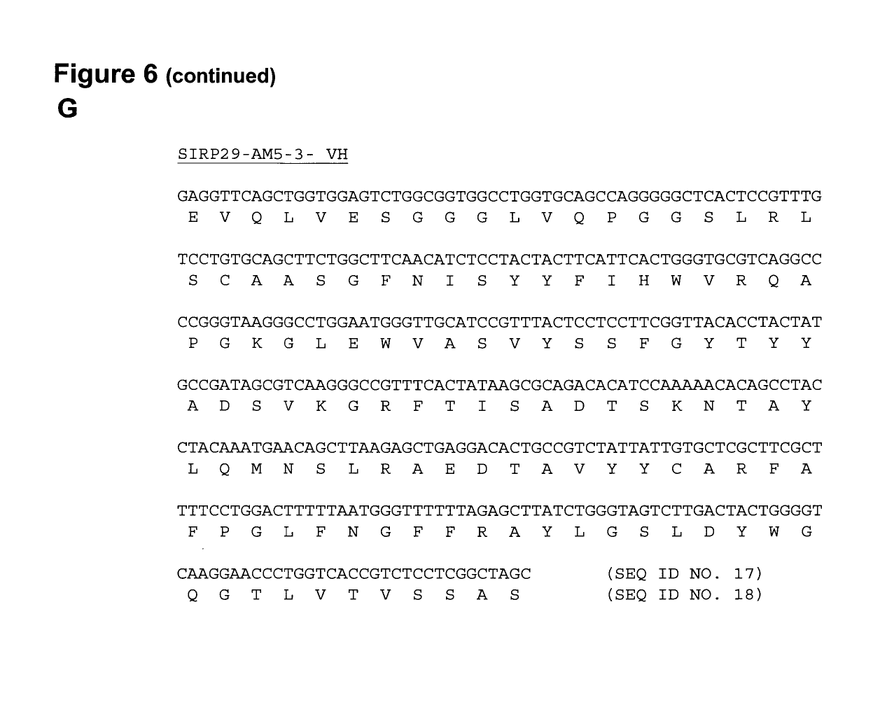

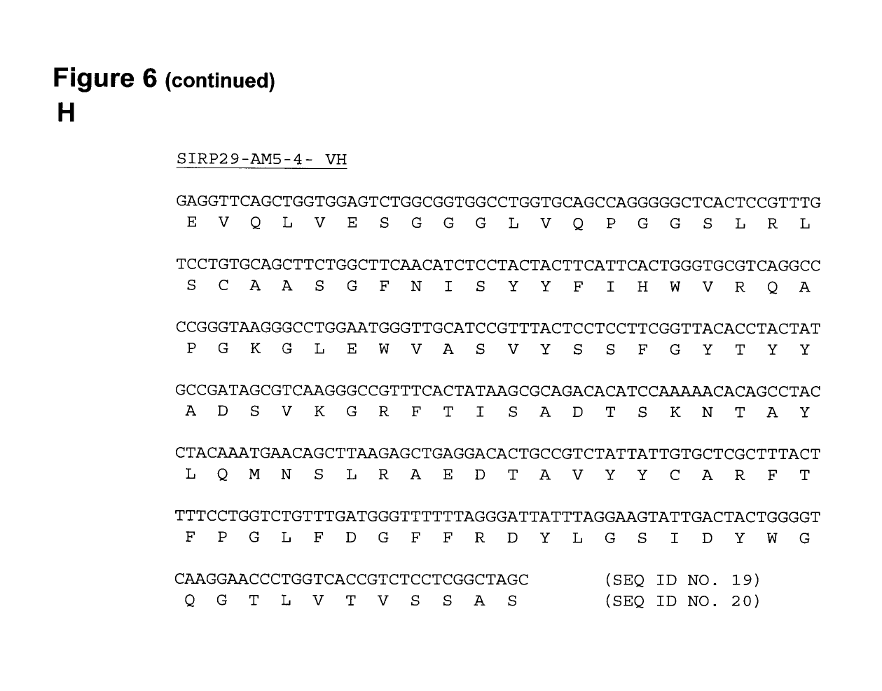

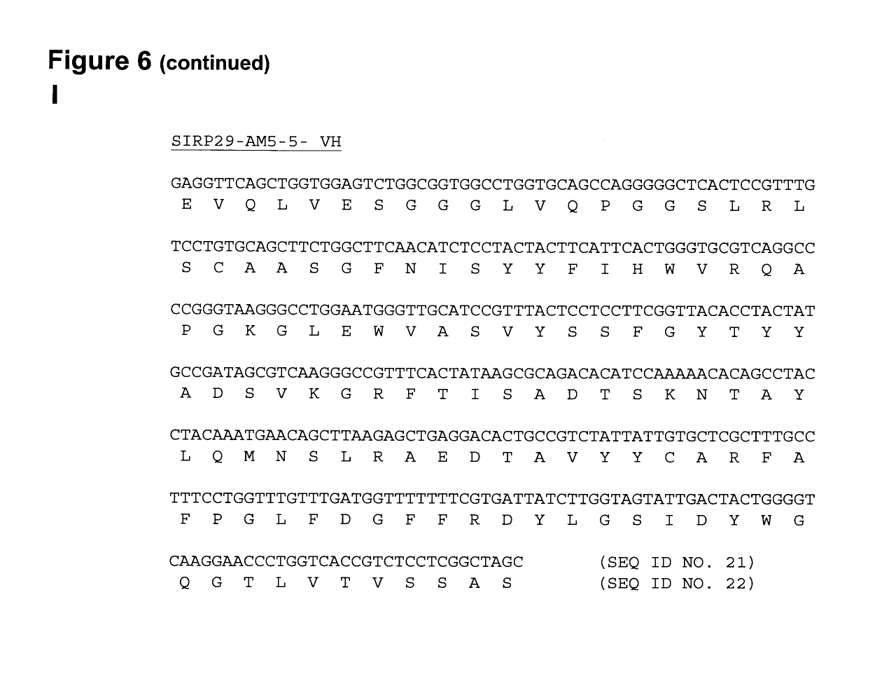

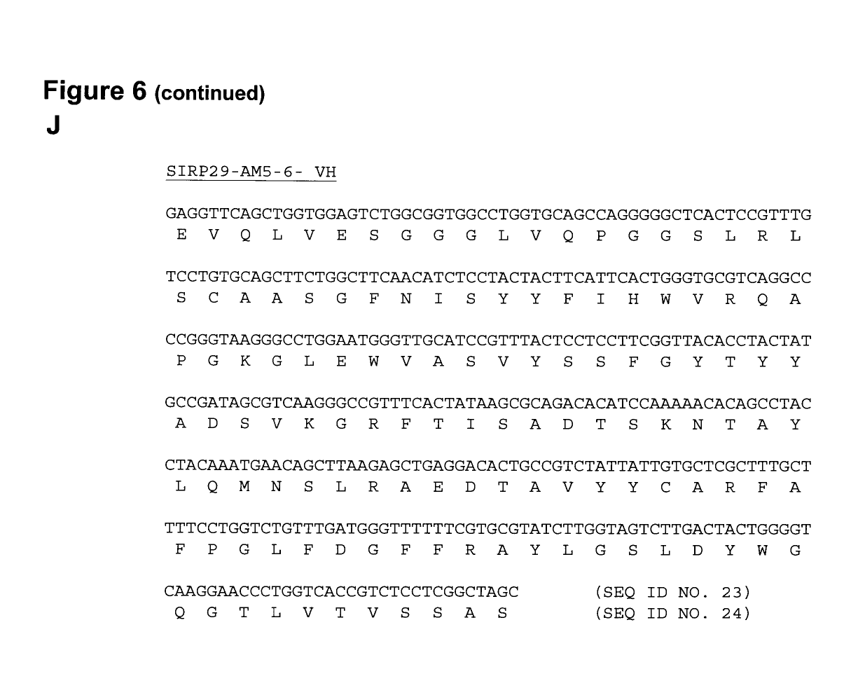

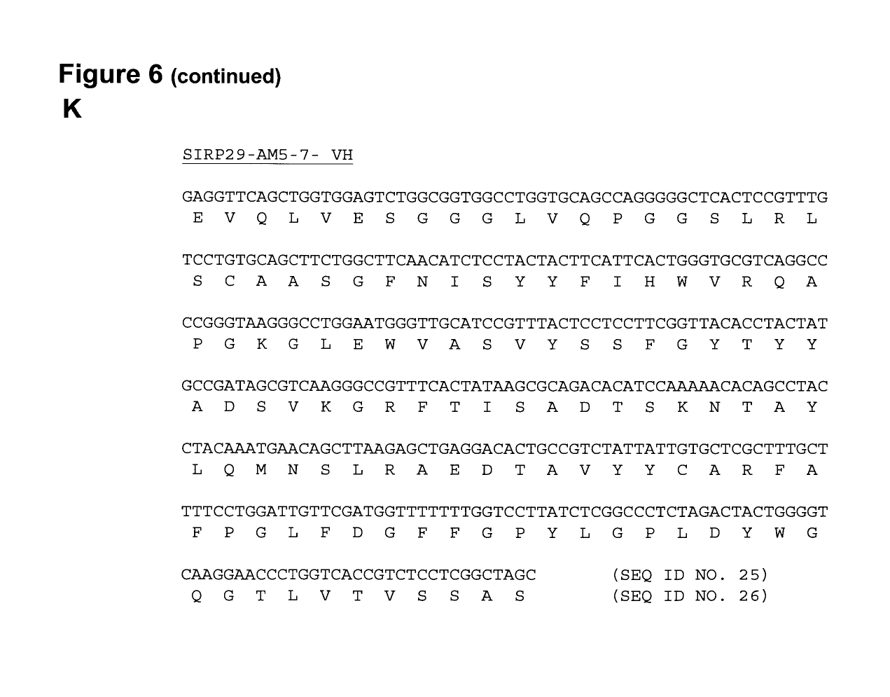

The nucleotide and translated amino acid sequences of SIRP29-AM3-35, SIRP 29-AM4-1 and SIRP 29-AM4-5 are shown in FIG. 6.

### IgG Reformatting

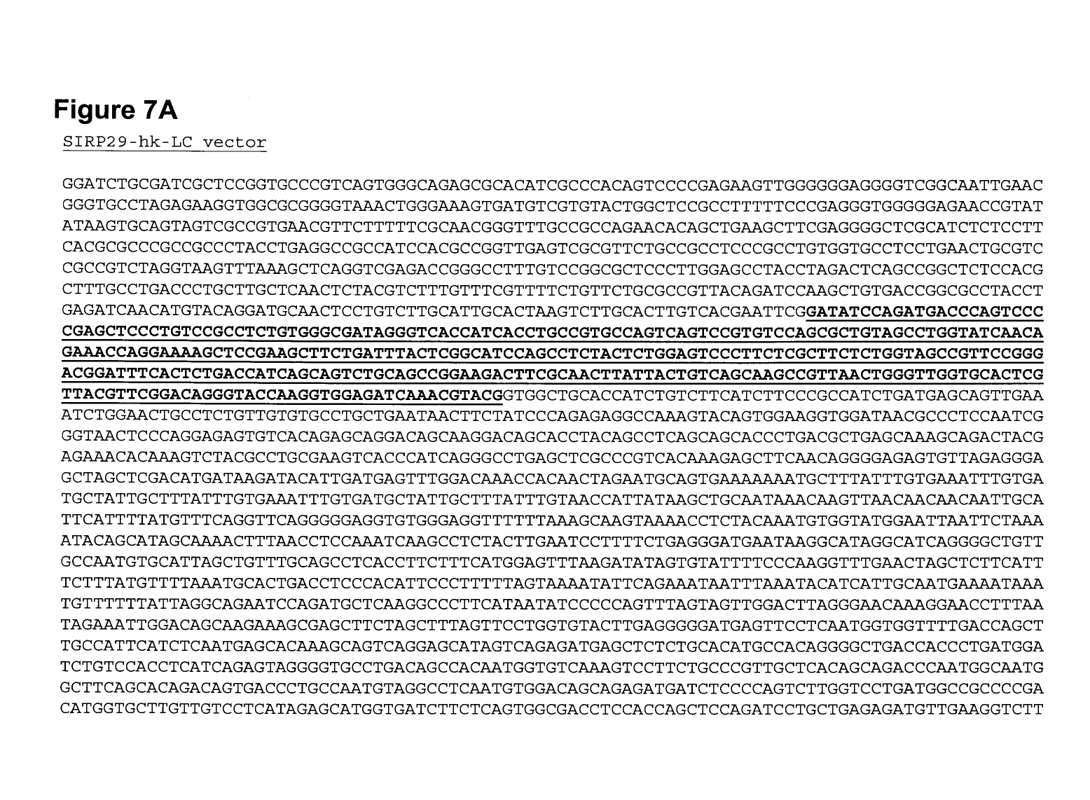

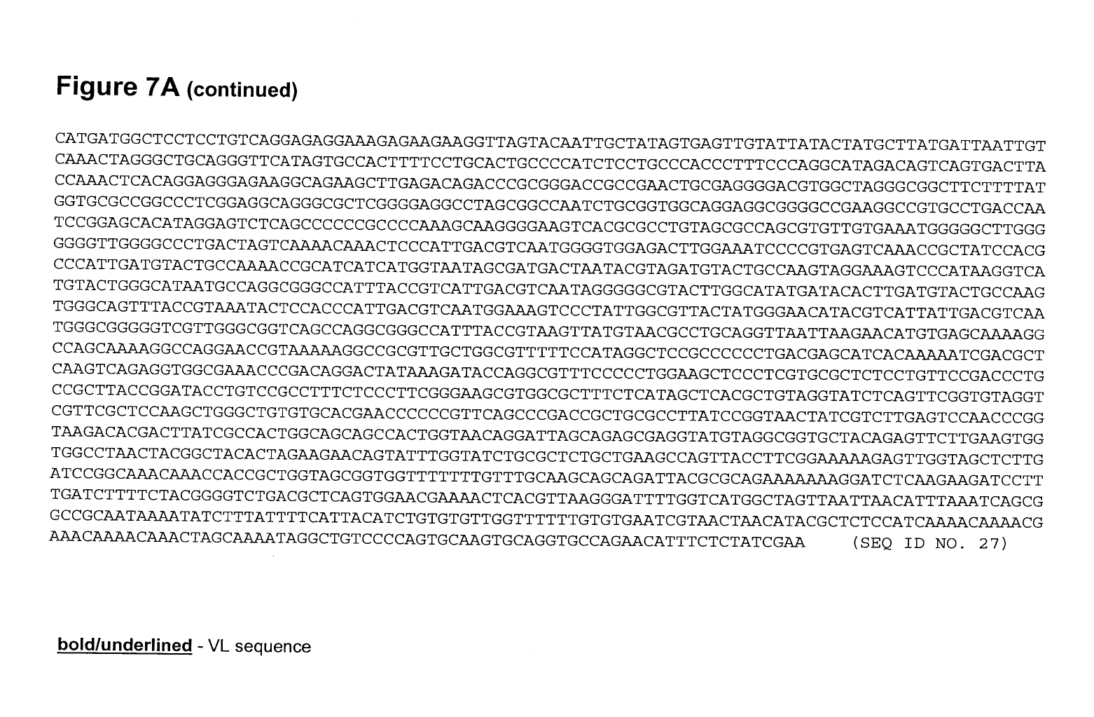

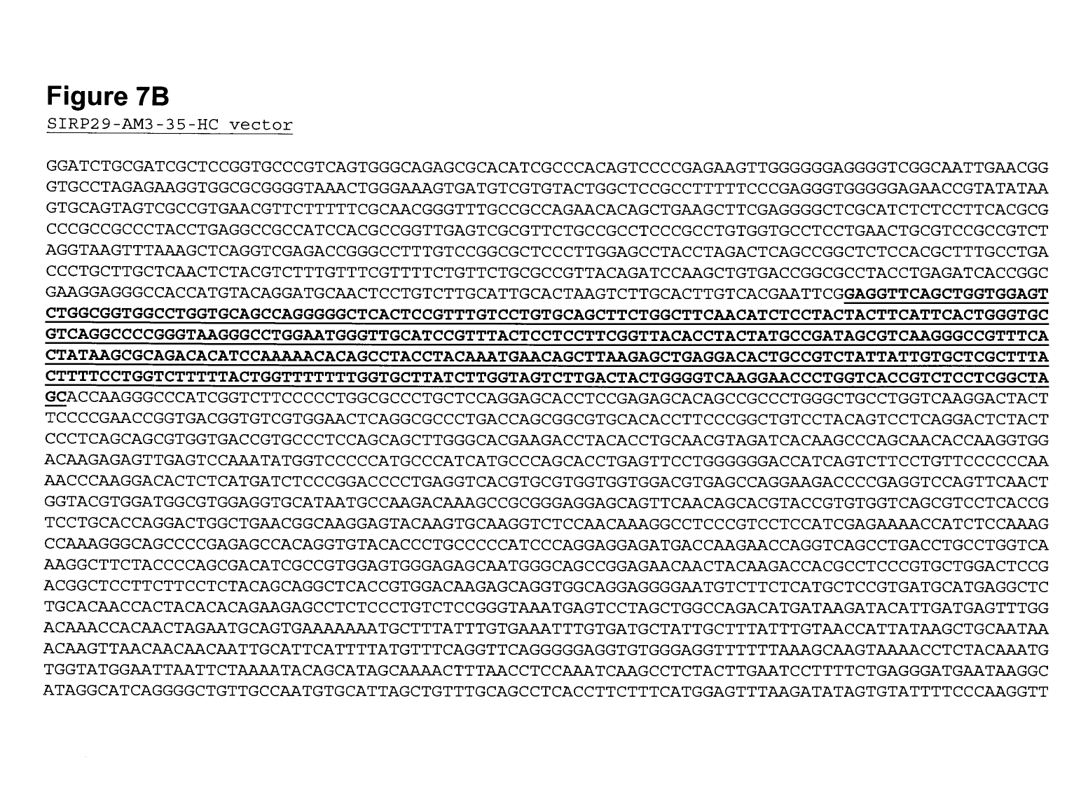

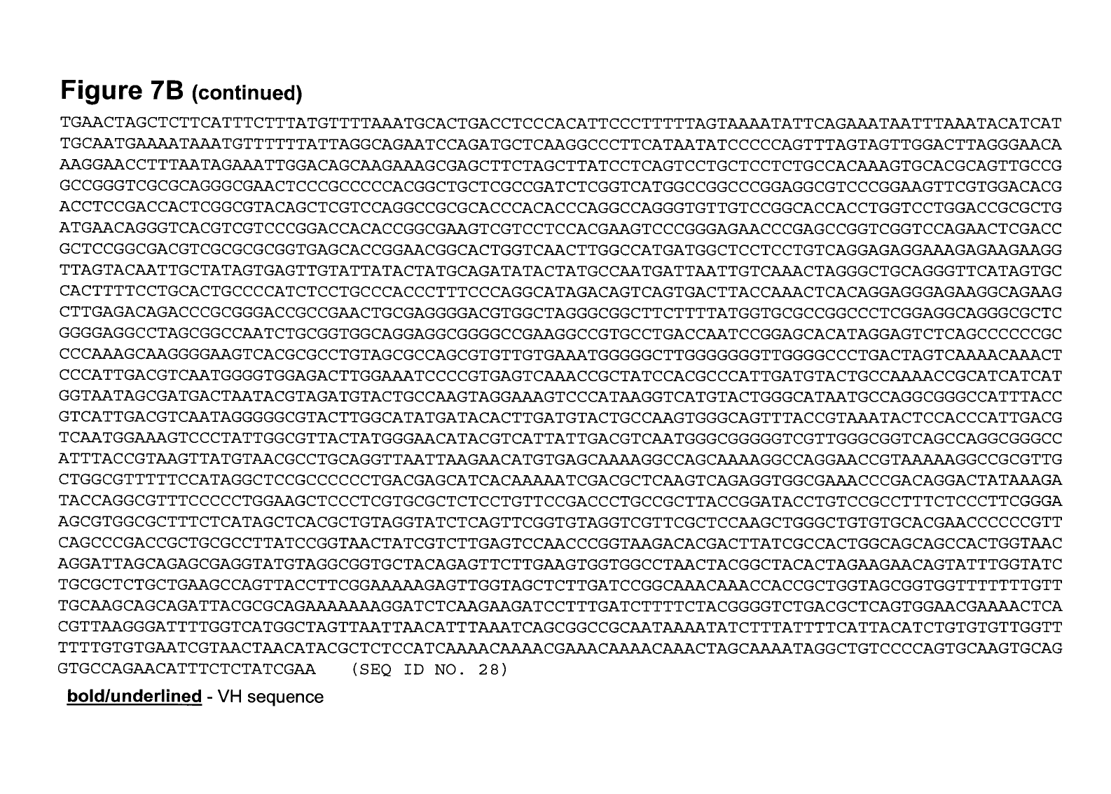

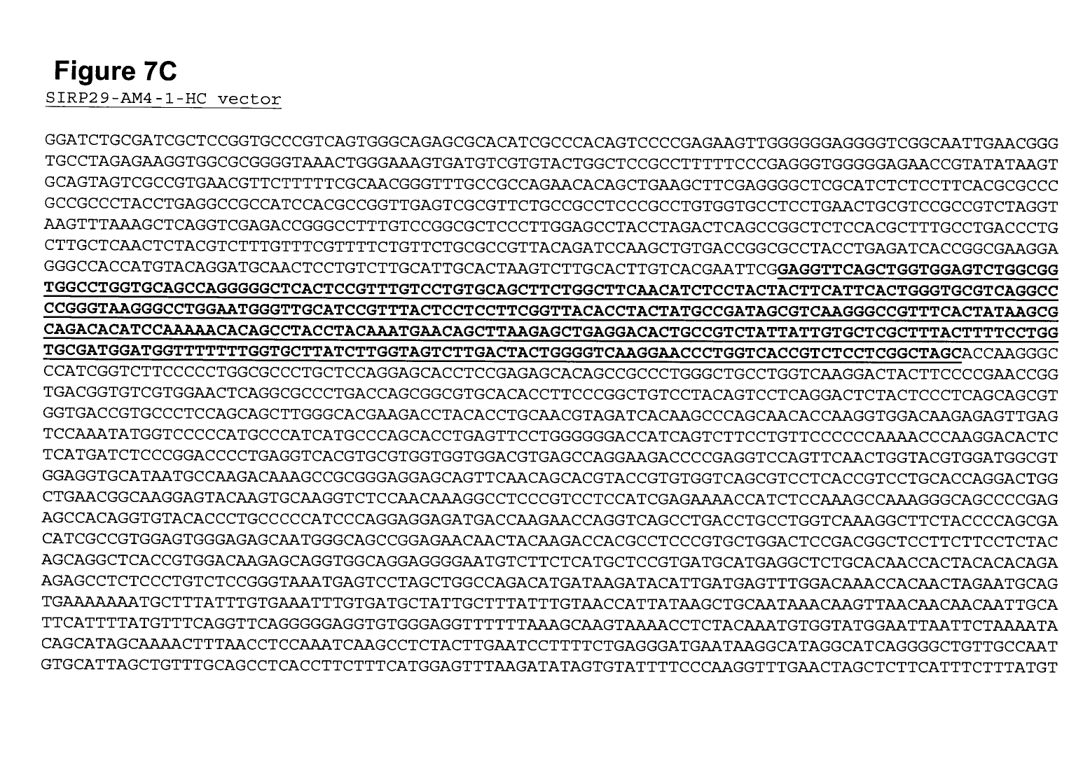

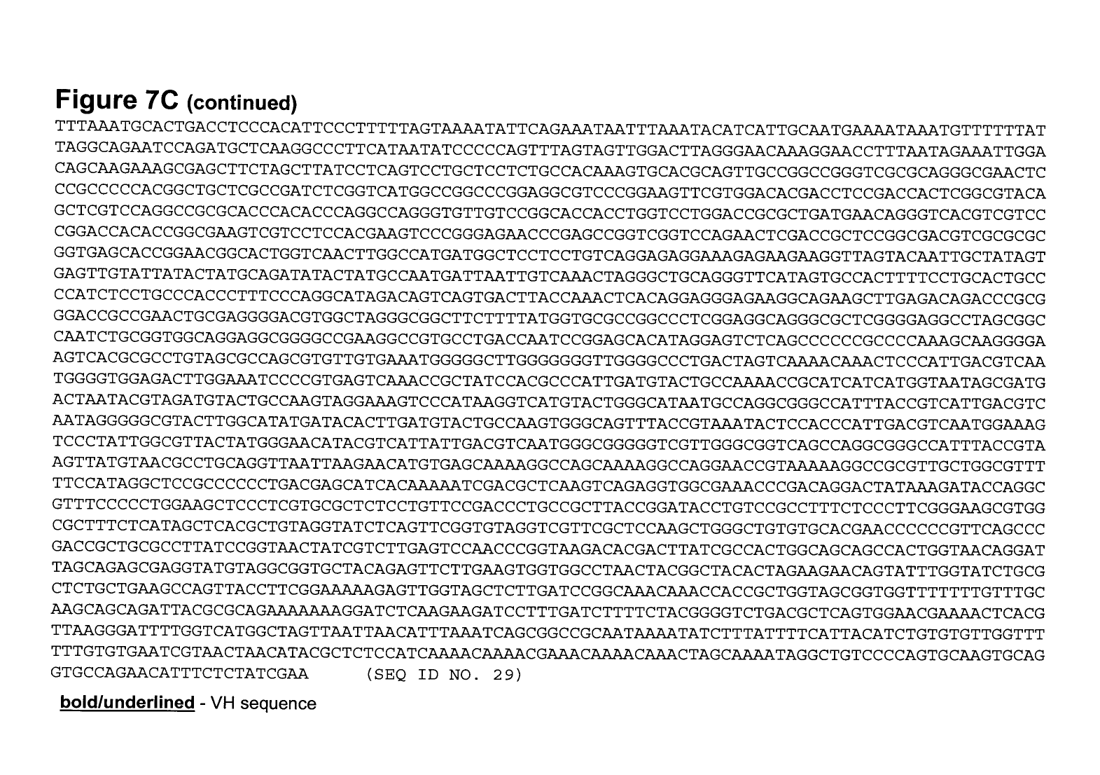

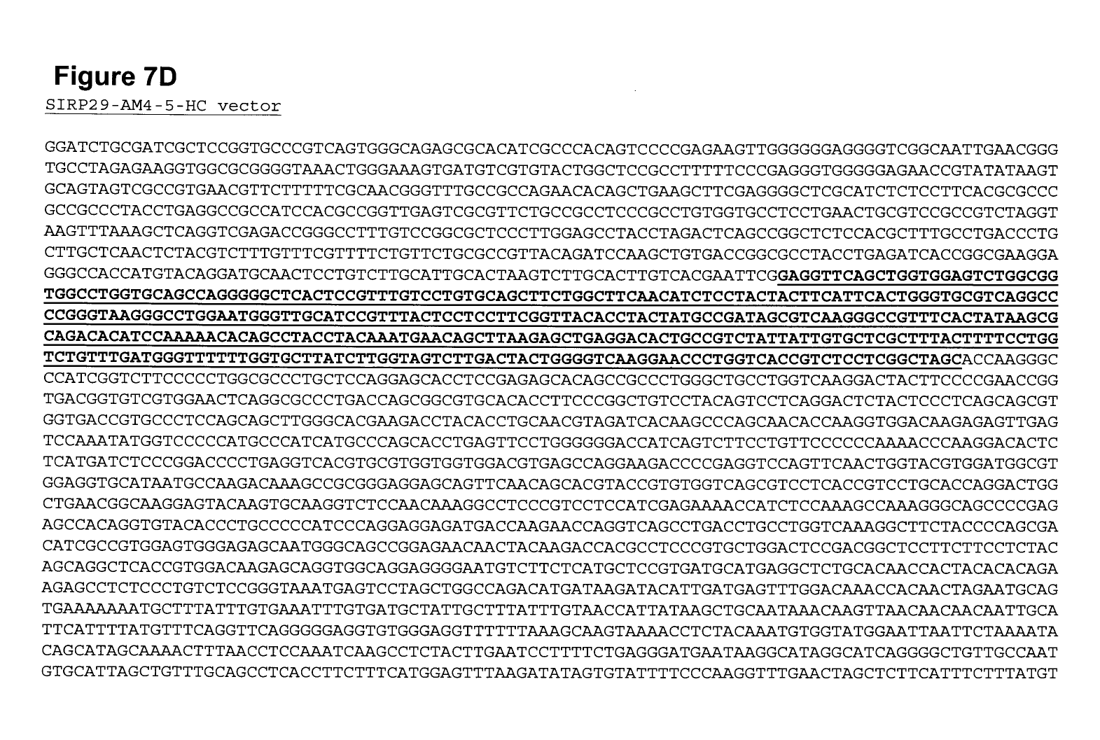

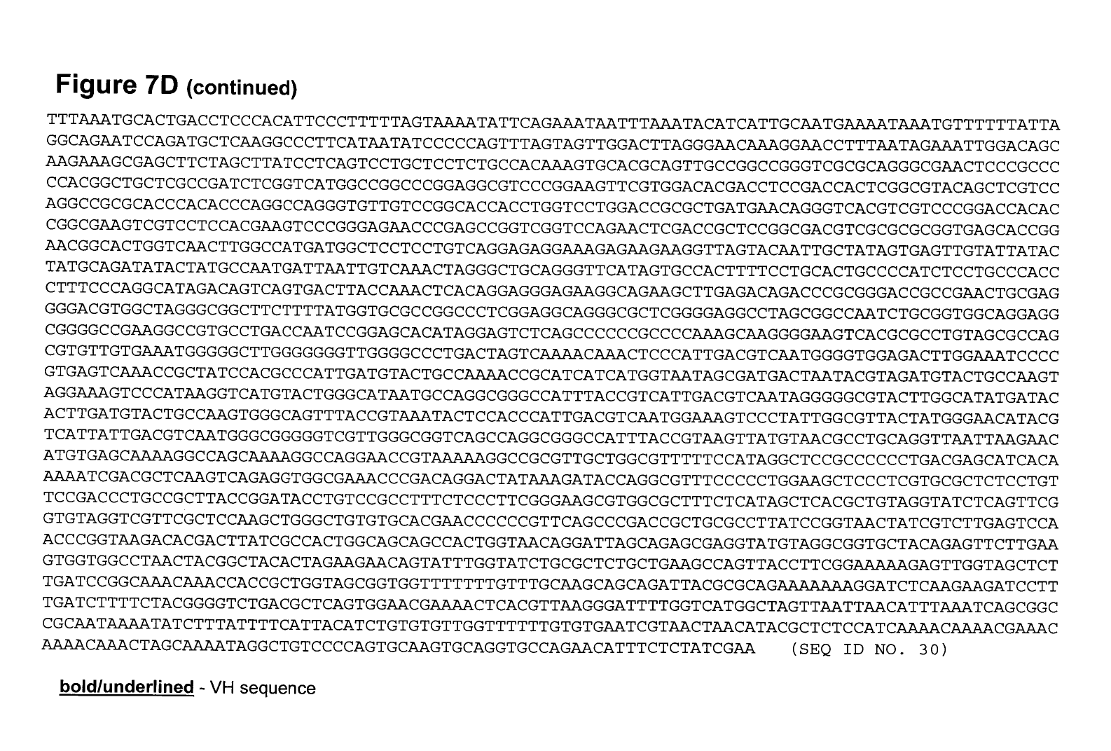

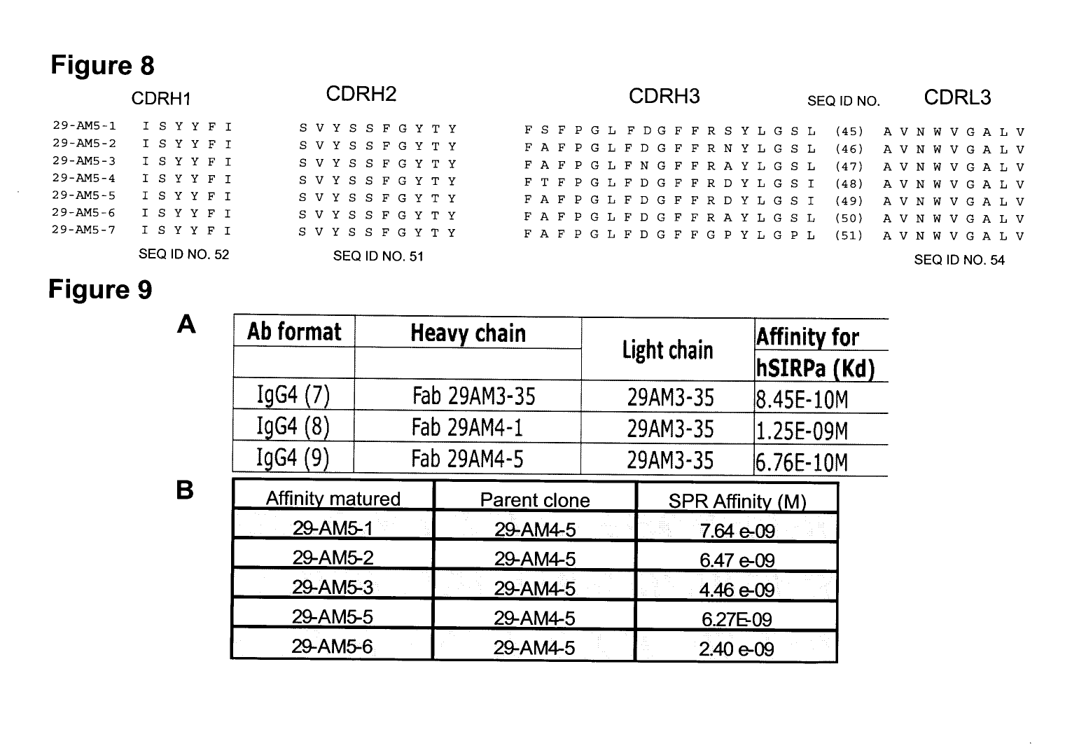

We reformatted SIRP29-AM3-35, SIRP 29-AM4-1 and SIRP 29-AM4-5 to produce full IgG versions by cloning the Fab into appropriate human IgG heavy chain encoding vectors wherein the Fab encodes the antigen combining site and the vector sequences supply the constant regions required to produce an IgG4 heavy chain. We also prepared a SIRP29-hk-LC human Iv light chain vector. The sequences of the heavy and light chain vectors is shown in FIG. 7. Cell lines were prepared containing SIRP29-hk-LC+ SIRP29-AM3-35, SIRP29-hk-LC+ SIRP 29-AM4-1 and SIRP29-hk-LC+ SIRP 29-AM4-5 in order to produce and purify the reformatted anti-human SIRPα antibodies. Note that all sequences are of human origin.

### Affinity of Anti-SIRPα Fab for Purified SIRPα-Fc Fusion Proteins

The affinities of SIRP29-AM3-35, SIRP 29-AM4-1 and SIRP 29-AM4-5 Fab for human and NOD mouse SIRPα IgV domains were determined by surface plasmon resonance using our novel human SIRPα-Fc and NOD mouse SIRPα-Fc fusion proteins. Both SIRP29-AM4-1 and SIRP29-AM4-5 display low nM affinities for the human target (FIG. 9A).

### Affinity of Anti SIRPα Fab for Human SIRPα Expressed on Macrophages and the CHO Cell Line

We developed a colorimetric quantitative cell-based binding assay using soluble protein specific for human SIRPα IgV.

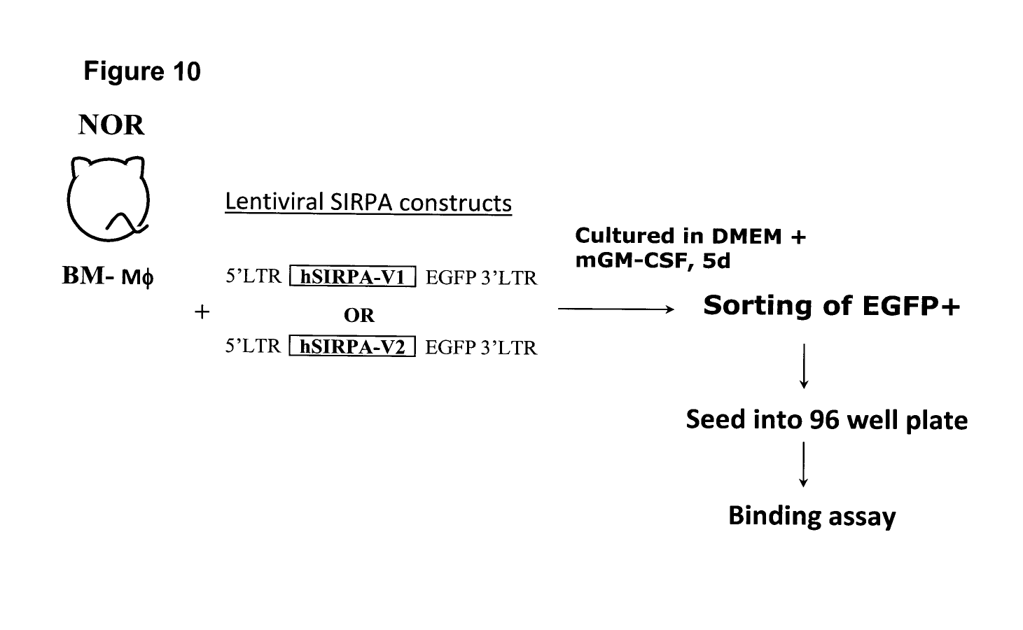

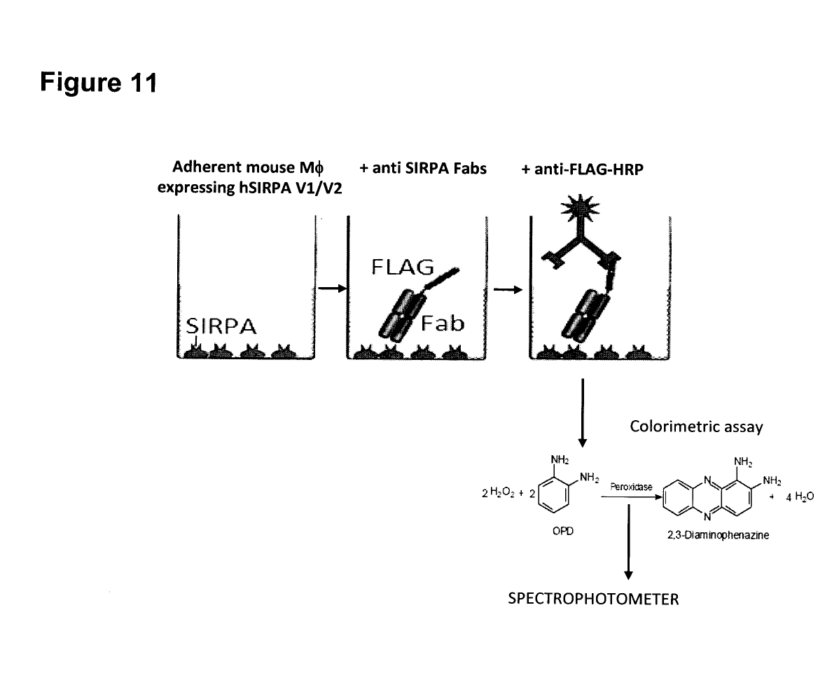

We prepared lentiviral vectors containing either human SIRPα V1 or SIRPα V2 IgV domains and the gene ecoding EGFP. Lentiviruses were produced in appropriate packaging cell lines, tited and used to infect either primary macrophages derived from the NOR mouse strain, or a CHO cell line. The infected cells were selected for EGFP expression by cell sorting (FIG. 10) and used in the binding assay shown in FIG. 11.

Infected macrophages expressing human SIRPα proteins were seeded in a 96-well plate and incubated with Fab 35 or human CD47-Fc fusion proteins for 30 min at 37° C. After washing, wells were incubated with HRP-conjugated goat polyclonal anti-human Fc antibody to detect hCD47-Fc binding or with HRP-conjugated mouse monoclonal anti-FLAG antibody to detect Fab 35 binding. Binding was detected by assaying HRP activity using the substrate 3,3′,5,5′-tetramethylbenzidine (TMB). The analysis of the data and the generation of the binding curves were performed using PRISM ver. 4.0, GraphPad software. Each data point represents specific binding, which was computed by subtracting nonspecific binding to NOR macrophages infected with empty lentivirus.

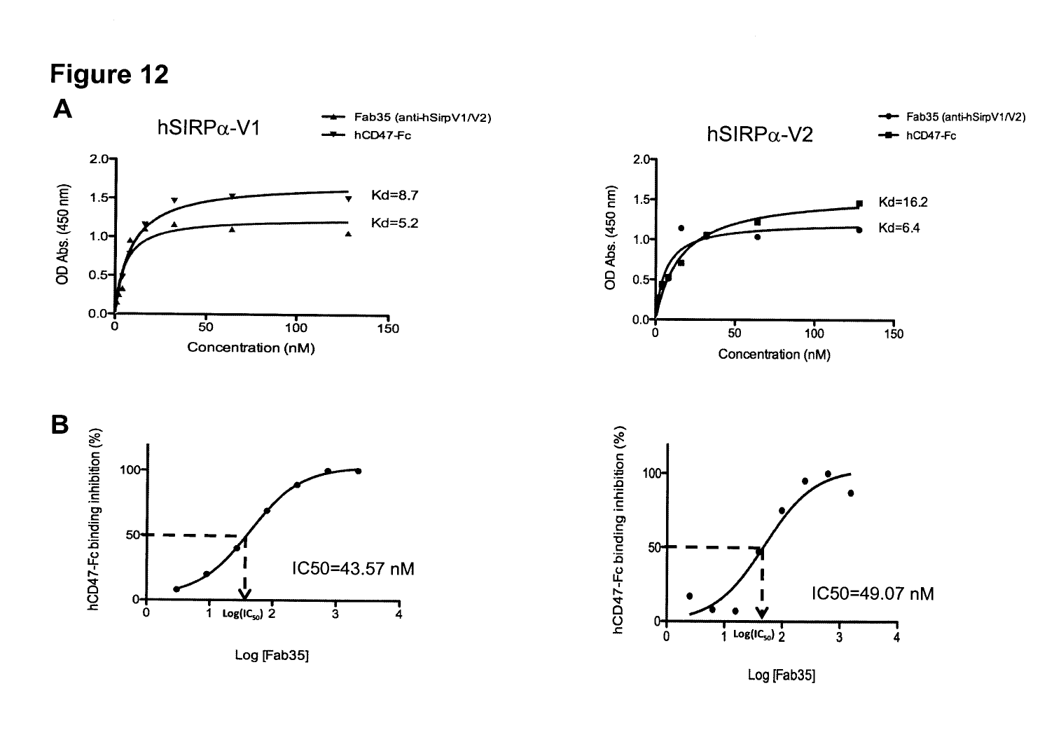

SIRP29-AM3-35 displayed low nM affinity for both of the most common IgV region variants of human SIRPα expressed on the surface of NOR macrophages, and compared favourably to the binding affinity of CD47-Fc for human SIRPα (FIG. 12A left SIRPα-V1, FIG. 12A right SIRPα-V2). NOR macrophages expressing human SIRPα variants V1 (FIG. 12 left panels) or V2 (FIG. 12 right panels) were incubated with escalating concentrations of hCD47-Fc or SIRP29-AM3-35 (Fab35) for 45 min at 37° C. (FIG. 12). After washing, HRP-conjugated goat polyclonal anti-human Fc antibody was added to detect human CD47-Fc binding. IC50 for Fab 35 binding to SIRPα-V1 (FIG. 12 B left) and SIRPα-V2 (FIG. 12 B right) were calculated from inhibition dose response curves. Data analysis was performed using PRISM v. 4.0 GraphPad.

### SIRP29-AM3-35 and Affinity Matured AM4-5 and AM4-1 Antibodies Inhibit CD47 Binding to Human SIRPα Expressed on Cells

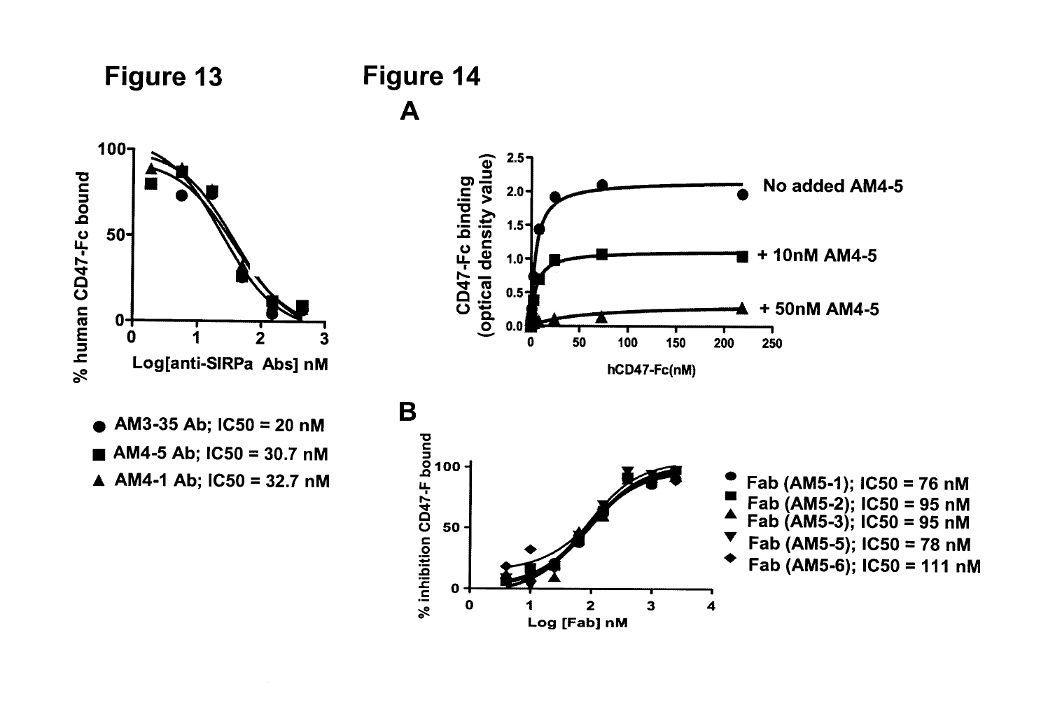

The binding assay described in FIG. 11 was used to evaluate the ability of antibody formatted versions of SIRPα-AM3-35, and further affinity matured antibodies AM4-5 and AM4-1 to inhibit the binding of CD47 to SIRPα expressed on the surface of macrophages (FIG. 13).

NOR macrophages expressing human SIRPα V2 were incubated with 25 nM hCD47-Fc either with or without escalating concentrations of AM3-35, AM4-5 or AM4-1 for 45 min at 37° C. (FIG. 13). After washing, a HRP-conjugated goat polyclonal anti-human Fc antibody was added to detect human CD47-Fc binding. IC50 for the three anti human SIRPα Ab were calculated and ranged from 20 nM-32.7 nM) from inhibition dose response curves. These IC50 values demonstrated the ability of these anti-SIRPα Abs to block engagement of SIRPα by CD47.

### SIRPα Ab AM4-5 Inhibits CD47 Binding to Human SIRPα Expressed on CHO Cells

Using the same assay described above (FIGS. 12 and 13), we examined SIRP29-AM4-5 inhibition of CD47 binding to human SIRPα (FIG. 14A). Dose response curves were generated in the absence of, or with addition of 10 nM or 50 nM concentrations of the Ab. CHO cells expressing SIRPα (V1) were incubated with increasing concentrations of CD47-Fc either in the absence (circle symbols) or in the presence of 10 nM (square symbols) or 50 nM (triangle symbols) of anti-SIRPα AM4-5 Ab for 45 min at 37° C. After washing, the cells were incubated with HRP-conjugated goat polyclonal anti-human Fc antibody to detect hCD47-Fc binding as previously described. Each data point represents specific binding computed by subtracting nonspecific binding to CHO cells infected with an empty lentivirus.

### Fourth Round Affinity Maturation

In a further approach, all residues in CDRH3 were soft-randomized, i.e. doped oligonucleotides were used such that each residue remains wild-type 50% of the time and can vary, as the rest of the other 19 amino acids, the remaining 50% of the time. This approach does not concentrate all the mutation in one structurally important region as in the previous round. The nucleotide sequence was replaced with following sequences for doping

A replaced with N1 (a mix of 70% A, 10% C, 10% G, 10% T)
C replaced with N2 (a mix of 10% A, 70% C, 10% G, 10% T)
G replaced with N3 (a mix of 10% A, 10% C, 70% G, 10% T)
T replaced with N4 (a mix of 10% A, 10% C, 10% G, 70% T)

A stop-template was made by inserting a stop codon in CDRH3 of 29-AM3-35 (the rest of the loops have same sequence as in AM4 clones). Three mutagenic oligonucleotides encoding for CDRH3 of 29-AM4-1, 4 and 5 were used to make a pooled library using the stop template for mutagenesis. A library of 3.5×10⁹ pooled diversity was generated and three different selections were done as follows:

| | | antig. | | |
|---|---|---|---|---|
| | | conc. | | |
| | Antigen | (μg/ml) | Washes | Pre-absorbtion |
| SIRP 1 | | | | |
| Round 1 | hSIRPαV2-Fc | 5 | 8 | SAV (10 μg/ml), 1-2 h |
| Round 2 | NOD SIRPα | 2 | 8 | NAV (10 μg/ml), 1-2 h |
| Round 3 | hSIRPαV2-Fc | 2 | 8 | SAV (10 μg/ml), 1-2 h |
| Round 4 | NOD SIRPα | 2 | 10 | NAV (10 μg/ml), 1-2 h |
| SIRP 2 | | | | |
| Round 1 | hSIRPαV2-Fc | 5 | 8 | SAV (10 μg/ml), 1-2 h |
| Round 2 | hSIRPαV2-Fc | 2 | 8 | NAV (10 μg/ml), 1-2 h |
| Round 3 | hSIRPαV2-Fc | 2 | 10 | SAV (10 μg/ml), 1-2 h |
| Round 4 | hSIRPαV2-Fc | 2 | 10 | NAV (10 μg/ml), 1-2 h |
| SIRP 3 | | | | |
| Round 1 | NOD SIRPα | 5 | 8 | SAV (10 μg/ml), 1-2 h |
| Round 2 | NOD SIRPα | 2 | 8 | NAV (10 μg/ml), 1-2 h |
| Round 3 | NOD SIRPα | 2 | 8 | SAV (10 μg/ml), 1-2 h |
| Round 4 | NOD SIRPα | 2 | 10 | NAV (10 μg/ml), 1-2 h |

The first two selections SIRP1 and SIRP2 generated a lot of positives while SIRP3 generated 4 hits.

| | | SEQ |
|---|---|---|
| Selected Sequences from Round4 | | ID |
| affinity maturation | | No |
| A2 | F S F P G L F D G F F S S Y L G S L | 67 |
| A3 | F T F P G L F D G F F G S Y L G S F | 68 |
| A4 | F T F P G L F D G F F R A Y L G S L | 69 |
| A5 | F A F P G L F E G F F R G Y L G S I | 70 |
| A6 | F S F P G L F D G F F G T Y L G S L | 71 |
| A7 | F S F P G L F D G F F R S Y L G S L | 72 |
| A8 | F T F P G L F N G F F G E Y L G S L | 73 |
| A9 | F A F P G L F D G F F R N Y L G S L | 74 |
| A10 | F A F P G L F D G F F A A Y L G S L | 75 |
| B1 | F S F P G M F D G F F G A Y L G S L | 76 |
| D1 | F S F P G L F D G F F G A Y L G S L | 77 |
| B5 | F A F P G L F D G F F G A Y L G S L | 78 |
| C10 | F A F P G Q F D G F F G A Y L G S L | 79 |
| C11 | F S F P G L F D G F F G A Y L G S I | 80 |
| B9 | F A F P G L F D G F F G A Y L G S I | 81 |
| B11 | F T L P G L I N G F F G A Y H G S L | 82 |
| D11 | F T F P G L F N G F F G A Y L G S L | 83 |
| C4 | F T F P G R F D G F F G A Y L G S I | 84 |
| D8 | Y T F P G L F D G F F G A Y L G S L | 85 |
| D12 | F T F P G L F D G F F G A Y L G S L | 86 |
| B7 | F S F P G L F D G F F R A Y L G S L | 87 |
| B6 | F A F P G L F N G F F R A Y L G S L | 88 |
| B12 | F A F P G L F D G F F R A Y L G S L | 89 |
| B3 | F T F P G L F D G F F S A Y L G S L | 90 |
| C1 | F A F P G L F D G F F A E Y L G S L | 91 |
| C2 | F T F P G L F D G F F G V Y L G S I | 92 |
| C3 | F T L P G L F S G F F G Y Y L G S L | 93 |
| C5 | F T F P G L F D G F F R D Y L G S I | 94 |
| D5 | F T L P G L L D G F F R D Y I G S L | 95 |
| C6 | F S F P G L F D G F F G G F L G S L | 96 |
| C7 | F S F P G L F D G F F G D Y L G S L | 97 |
| C9 | F T F P G L F D G F F G D Y L G S L | 98 |
| D2 | F S V P G L F D G F F R D Y L G S L | 99 |
| D4 | F A F P G L F E G F F G G Y L G S I | 100 |
| D6 | F T F P G L F D G F F G I Y L G S L | 101 |
| D7 | F S F P G K F D G F F G S Y L G S I | 102 |
| D9 | F A F P G L F D G F F S V F L G S L | 103 |
| E1 | F A F P G L F D G F F G A Y L G S I | 104 |
| F2 | F A F P G L F D G F F G A Y L G S L | 105 |
| F8 | F A F P G L F D G F F R D Y L G S I | 106 |
| G11 | F A F P G L F D G F F R A Y L G S L | 107 |
| E6 | F T F P G M F D G F F R A Y L G S L | 108 |
| E2 | F T F P G L F V G F F G A Y L G S L | 109 |
| E3 | F T F P G Q F H G F F G D Y L G S L | 110 |
| E5 | F T F P G Q F D G F F G P Y L G S L | 111 |
| H7 | F S F P G Q F D G F F G A Y L G S L | 112 |
| F7 | F T F P G Q F N G F F G A Y L G S L | 113 |
| E7 | F T F P G L F D G F F G S Y L G S L | 114 |
| F6 | F T F P G L F G G F F R S Y L G S L | 115 |
| E8 | F T F P G L F G G F F S D Y L G S L | 116 |
| E10 | F T F P G L F E G F Y R D Y L G S L | 117 |
| H3 | F A F P G M F D G F F G D Y L G S L | 118 |
| F1 | F T F P G L F D G F F R D F L G S L | 119 |
| E9 | F S S P G V F A G F F G A Y I G S L | 120 |
| E11 | F T F P G L F G G F F G A Y L G S L | 121 |
| F3 | S T V P G L F D G F F G A Y H G S L | 122 |
| F5 | Y A F P G L F D G F F G A Y L G S L | 123 |
| F9 | F T F P G R F D G F F G A Y L G S I | 124 |
| F10 | F T F P G R F D G F F G A Y L G S L | 125 |
| F12 | F S F P G L F G G F F R A D L G S L | 126 |
| G1 | F T F P G L F N G F F G A Y L G S L | 127 |
| G2 | F A F P G T F S G F Y G A F L G S I | 128 |
| G3 | F T F P G L F S G F F G A Y L G S L | 129 |
| G4 | F S F P G L F N G F F G A Y I G S I | 130 |
| G5 | F T F P G L L H G F Y G T Y I G S L | 131 |
| G6 | Y T F P G L F D G F F G K Y L G S L | 132 |
| G8 | F S F P G M F D G F F G A Y L G S L | 133 |
| G12 | F T F P G L F D G F F S A Y L G S L | 134 |
| H2 | F T F P G L F G G F F G G Y L G S L | 135 |
| H5 | Y S F P G L F D G F F G A Y L G S L | 136 |
| H6 | F T F P G L F A G F F G A Y L G S L | 137 |
| H10 | F S F P G L F H G F F G A Y L G S L | 138 |
| H11 | F A F P G L F D G F F G P Y L G P L | 139 |

### SIRPα Fab AM5-1, 5-2, 5-3, 5-5, 5-6 Inhibit CD47 Binding to Human SIRPα Expressed on CHO Cells

Fab obtained following an additional round of affinity maturation were examined for their ability to inhibit interaction between human CD47-Fc and human SIRPα V2 expressed on the surface of CHO cells using the same assay described above (FIG. 14 B). Dose response curves for binding of hCD47-Fc to CHO cells expressing human SIRPα V2 were generated in the absence of, or with escalating concentrations of Fab AM5-1 (circle symbol), AM5-2 (square symbol), AM5-3 (upward triangle symbol), AM5-5 (downward triangle symbol) and AM5-6 (diamond symbol). Each data point represents specific hCD47-Fc binding. IC50 values were calculated from these binding data (range 76-111 nM). These results demonstrate that the fourth round affinity maturation Fab compounds display potent inhibition of binding between CD47 and SIRPα expressed on cells.

### SIRPα Ab AM4-5 Inhibits the Growth and Migration of Primary Human AML Cell In Vivo

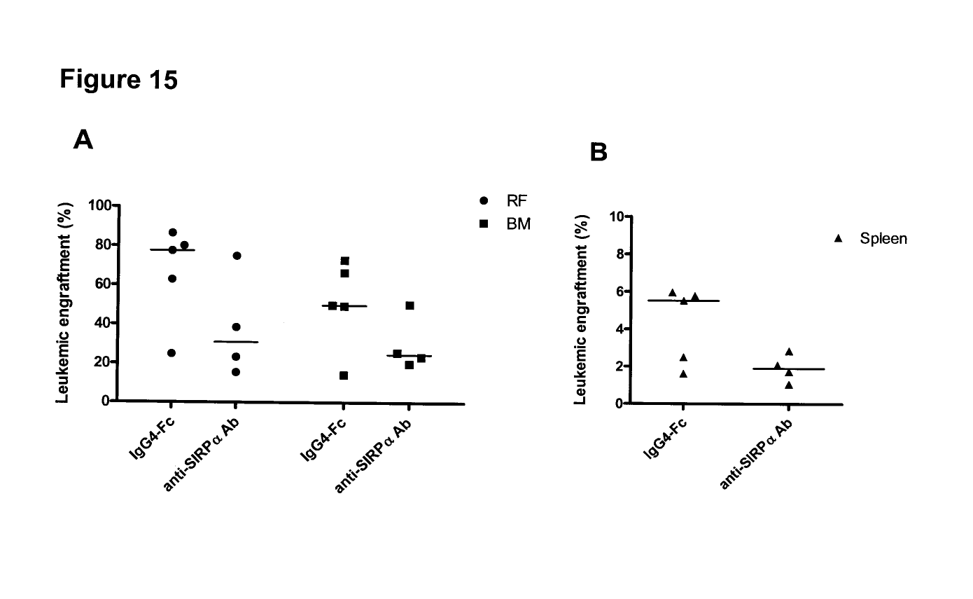

Xenotransplantation into immune-deficient NOD.SCID.γC^{-/-} (NSG) mice is the best available quantitative in vivo assay to evaluate the biology of primary human normal hematopoeitic and leukemia cells. This xenotransplantation assay was used to evaluate the impact of SIRPα Ab AM4-5 on the engraftment and dissemination of primary human AML cells (FIG. 15). Cohorts of NSG mice were transplanted with primary human AML cells by injection into the right femur (RF). The mice were left for 21 days to allow AML expansion and spread to other tissues. The mice were then treated with either anti-SIRPα Ab (AM4-5) or a matched control human IgG4-Fc protein, at 8 mg/kg, injected intra-peritoneally 3×/week for 4 weeks. The NSG mice were then sacrificed and analyzed for the percentage of human AML engraftment by multi-parameter flow cytometry using human-specific antibodies (anti-hCD33⁺ and hCD45⁺) in (λ) the injected RF (circle symbols) and non-injected bones (BM; other femur and tibias, square symbols) and in (B) the spleen (triangle symbols). Each symbol represents analysis of that tissue from a single NSG mouse. These data indicate that SIRPα Ab AM4-5 reduced the engraftment and dissemination of a primary AML patient sample, suggesting that this approach may display therapeutic efficacy against leukemia in vivo.

Using 29-AM4-5 as the baseline sequence, analysis of all affinity maturation rounds reveals the following sequence and possible amino acid substitutions, predicted to have binding affinity to SIRPα, albeit with possible lower affinity for certain substitutions (particularly at positions 1, 3, 11 and 18).

```
29-AM4-5
F T F P G L F D G F F G A Y L G S L
Y A Y H H E Y R S F H A F
    S L F I N A T H Y G I
        V M L A S G I P
                    Q M S T D
                    R T E
                    V G K
                    K Y
                    T N
                    A P
```

Although preferred embodiments of the invention have been described herein, it will be understood by those skilled in the art that variations may be made thereto without departing from the spirit of the invention or the scope of the appended claims. All documents disclosed herein are incorporated by reference.

1. An antibody comprising at least one CDR selected from the group consisting of:
a) CDRL1:
(SEQ ID NO. 55)
S-V-S-S-A;
b) CDRL2:
(SEQ ID NO. 56)
S-A-S-S-L-Y-S;
c) CDRL3:
(SEQ ID NO. 54)
A-V-N-W-V-G-A-L-V;
d) CDRH1:
(SEQ ID NO. 52)
I-S-Y-Y-F-I;
e) CDRH2:
(SEQ ID NO. 53)
S-V-Y-S-S-F-G-Y-T-Y;
and
f) CDRH3:
X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈;
wherein:
X₁ is F;
X₂ is T, A or S;
X₃ is F;
X₄ is P;
X₅ is G;
X₆ is L, H, F, M, Q, R, V, K, T or A;
X₇ is F, H, I, L or M;
X₈ is D, E, N, A, S, T or G;
X₉ is G;
X₁₀ is F;
X₁₁ is F;
X₁₂ is G, R, A, S or T;
X₁₃ is A, S, T, G, D, E, K, Y, N or P;
X₁₄ is Y, F or H;
X₁₅ is L, H, Y or I;
X₁₆ is G;
X₁₇ is S, A, G or P; and
X₁₈ is L.

2. (canceled)

3. The antibody of claim 1, wherein CDRH3 is:
(SEQ ID NO. 141)
F-T-F-P-G-A-F-T-G-F-F-G-A-Y-L-G-S-L.

4. The antibody of claim 1, wherein CDRH3 is:
(SEQ ID NO. 39)
F-T-F-P-G-A-M-D-G-F-F-G-A-Y-L-G-S-L.

5. The antibody of claim 1, wherein CDRH3 is:
(SEQ ID NO. 42)
F-T-F-P-G-D-F-R-G-F-F-G-A-Y-L-G-S-L.

6. The antibody of claim 1, wherein CDRH3 is:
(SEQ ID NO. 43)
F-T-F-P-G-L-F-D-G-F-F-G-A-Y-L-G-S-L.

7. The antibody of claim 1, wherein CDRH3 is:
(SEQ ID NO. 45)
F-S-F-P-G-L-F-D-G-F-F-R-S-Y-L-G-S-L.

8. The antibody of claim 1, wherein CDRH3 is:
(SEQ ID NO. 46)
F-A-F-P-G-L-F-D-G-F-F-R-NS-Y-L-G-S-L.

9. The antibody of claim 1, wherein CDRH3 is:
(SEQ ID NO. 47)
F-A-F-P-G-L-F-N-G-F-F-R-A-Y-L-G-S-L.

10. The antibody of claim 1, wherein CDRH3 is:
(SEQ ID NO. 48)
F-T-F-P-G-L-F-D-G-F-F-R-D-Y-L-G-S-I.

11. The antibody of claim 1, wherein CDRH3 is:
(SEQ ID NO. 49)
F-A-F-P-G-L-F-D-G-F-F-R-D-Y-L-G-S-I.

12. The antibody of claim 1, wherein CDRH3 is:
(SEQ ID NO. 50)
F-A-F-P-G-L-F-D-G-F-F-R-A-Y-L-G-S-L.

13. The antibody of claim 1, wherein CDRH3 is:
(SEQ ID NO. 51)
F-A-F-P-G-L-F-D-G-F-F-G-P-Y-L-G-P-L.

14. The antibody of claim 1, wherein the remaining residues in any portion of the light chain variable domain, of the antibody, comprises the corresponding residues from SEQ ID NO. 6.

15. The antibody of claim 1, wherein the remaining residues in any portion of the heavy chain variable domain, of the antibody, comprises the corresponding residues from SEQ ID NO. 8.

16. The antibody of claim 1, comprising at least CDRH1, CDRH2 and CDRH3.

17. The antibody of claim 1, comprising all of CDRL1, CDRL2, CDRL3, CDRH1, CDRH2 and CDRH3.

18. (canceled)

19. The antibody of claim 1, wherein the antibody is an antibody fragment.

20. A pharmaceutical composition comprising the antibody of claim 1 and a pharmaceutically acceptable carrier.

21.-22. (canceled)

23. A method of treating hematological cancer in a subject in need of treatment, the method comprising administering a therapeutically effective amount of the antibody of claim 1.

24.-26. (canceled)

27. The method of claim 23, wherein the hematological cancer is leukemia, preferably acute myeloid leukemia or acute lymphoblastic leukemia.
